(19) United States
(12) Patent Application Publication
(10) Pub. No.: US 2016/0235366 A1
(43) Pub. Date: Aug. 18, 2016

(54) Methods and Systems for Obtaining Clinical Samples

(71) Applicant: Theranos, Inc., Palo Alto, CA (US)

(72) Inventors: Elizabeth A. Holmes, Palo Alto, CA (US); Jeffrey Blickman, Palo Alto, CA (US)

(21) Appl. No.: 15/041,744

(22) Filed: Feb. 11, 2016

Related U.S. Application Data
(63) Continuation of application No. PCT/US2014/054625, filed on Sep. 8, 2014.
(60) Provisional application No. 61/875,092, filed on Sep. 8, 2013.
(60) Provisional application No. 61/894,166, filed on Oct. 22, 2013.

(51) Int. Cl. A61B 5/00; A61B 10/00; A61B 5/15

(52) U.S. Cl. CPC A61B 5/6889; A61B 5/1405; A61B 10/0096

(57) ABSTRACT

Methods for obtaining a sample from a subject include providing a sample collection room within a retail store; and obtaining a sample. Methods for analyzing a sample include providing a sample collection room within a retail store; obtaining a sample; and analyzing it, e.g., at the retail location. A waiting room; a bathroom; and a pass-through from bathroom to sample collection room may be provided. Sample collection rooms may include one or more of a warming table; a warming plate; a finger warmer; a fingertip warmer; an air-warmer; a reclining chair; a calming feature; and adjustable lighting. Samples may be analyzed at the retail location or sent to elsewhere for analysis. Sample collection rooms may house a sample analysis device or system. Samples may be small, e.g., a finger-stick, and may be analyzed in a short period of time, e.g., in less than five hours, or less than four hours.

### BACKGROUND

Clinical samples are useful in many situations, including being useful for monitoring the health of subjects, for diagnosing diseases or pathological conditions, and for monitoring the progress of therapeutic interventions, among various uses.

Obtaining clinical samples is required for such uses; however, subjects may find providing clinical samples, such as blood, urine, or tissue swab samples, to be painful, or difficult, or inconvenient. Accordingly, improves methods for obtaining clinical samples are needed.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

Improved methods for obtaining clinical samples include providing a designated location in a retail store where a clinical sample may be provided for analysis. In embodiments, improved methods for obtaining clinical samples include providing a designated location in a retail store, where the designated location is configured for obtaining a clinical sample; and obtaining a clinical sample from a subject. In embodiments, the sample may be analyzed at the same location; or at an adjacent location; or may be sent to a different location for analysis. In embodiments, the sample may be analyzed by an automatic analysis device or system at the same location; or at an adjacent location; or at a different location. A designated location in a retail store may be a sample collection room.

In embodiments, the designated location in the retail store comprises a room that is separate from other areas in the retail store; for example, the designated location may be separated from other areas in the retail store by a wall having a door, while being accessible to subjects from within the retail store. In embodiments, the designated location in the retail store comprises a room that is adjacent to other areas in the retail store, while being separate from other areas in the retail store; in embodiments, the designated location may be accessible to subjects from outside the retail store. In embodiments, the designated location in the retail store comprises a plurality of rooms that are separate from other areas in the retail store; for example, at least one of such rooms may be accessible directly from other areas in the retail store (e.g., via a door or entry-way), or at least one of such rooms may be adjacent the other areas of the retail store and be accessible from outside the retail store. For example, such rooms may include a waiting room, a sample collection room, a bathroom, a sample analysis room, and other rooms. In embodiments, a room may serve multiple functions (e.g., a bathroom may serve as a sample collection room where a urine or stool sample is collected; or a sample collection room may serve as a sample analysis room where a sample analysis device or a sample analysis system is located in the room where a sample is collected).

In embodiments, the designated location in the retail store may be placed at the back of the store (i.e., at a location spaced away from the main public entrance, or entrances, to the retail store). In embodiments, the designated location in the retail store may be placed near the front of the store (i.e., at a location close to the main public entrance, or entrances, to the retail store). In embodiments, the designated location in the retail store may be placed along, or near to, a lateral portion of the store (i.e., at a location to the right or to the left of the main public entrance, or entrances, to the retail store). In embodiments, the designated location in the retail store may be placed within the store away from the front, rear, or sides of the retail store.

In embodiments, the designated location in the retail store comprises at least two rooms. In embodiments, the designated location in the retail store comprises at least two rooms, wherein at least one room is configured to serve as a waiting room, and at least one room is configured for obtaining a sample from a subject; in embodiments, the at least one room configured for obtaining a sample from a subject is configured to provide privacy for a subject while a sample is obtained. In embodiments, at least two rooms may be connected by a pass-through (e.g., a passageway) connecting a sample collection room (e.g., a room configured for obtaining samples, such as blood samples) to a bathroom. In embodiments, at least two rooms may be connected by a pass-through (e.g., a passageway) connecting a room configured for obtaining samples to a room configured to serve as a waiting room. In embodiments, at least two rooms may be connected by a pass-through (e.g., a passageway) connecting a room configured for obtaining samples to a room configured to serve as a sample analysis room. In embodiments, such a pass-through or passageway may have a door or hatch having an open configuration and a closed configuration. Such a pass-through may have, for example, a round shape, a rectangular shape, or a square shape. Such a pass-through may have, for example, a door which may be closed when the pass-through is not in use for transfer of a sample from a patient to sample collection personnel. Such a door may be, for example, a sliding door (e.g., may open and close substantially in the plane of a wall through which the pass-through passes), or may be a hinged door.

In embodiments, a sample collection room may include a sample analysis device; in embodiments, such a sample analysis device may be configured to analyze a sample obtained in said sample collection room. In embodiments, a sample collection room may be adjacent, or near to, a location or room housing a sample analysis device; for example a sample analysis device may be located in a room adjacent to a sample collection room. In embodiments, a sample collection room may include a sample analysis device that is configured to analyze a fluid sample having a volume of about 500 μL or less obtained in the sample collection room. In embodiments, a sample collection room may be adjacent, or near to, a location or room housing a sample analysis device that is configured to analyze a fluid sample having a volume of about 500 μL or less obtained in the sample collection room.

In embodiments, a sample collection room may include a sample transport container configured to hold a sample, or a plurality of samples. In embodiments, a sample collection room may include a sample transport container configured to hold a sample container, or a plurality of sample containers, said sample containers configured to hold fluid samples having volumes of about 500 μL or less. In embodiments, a sample transport container may hold a sample container, or a plurality of sample containers, which include sample in one or more of the sample containers. In embodiments, a sample transport container may be insulated, effective to reduce the rate of change of temperature within the container. In embodiments, a sample transport container may be refrigerated, effective maintain a desired temperature within the container and in the samples held in the container.

In embodiments, a sample collection room may include a centrifuge. In embodiments, a centrifuge may be configured to receive and operate upon a small sample, or a plurality of samples, collected in the sample collection room, or to receive and operate upon a calibration container. In embodiments, a centrifuge included in a sample collection room may be configured for placement on, and for use on, a table, a counter-top, or other work surface.

In embodiments, a sample collection room may include a computer, a computer keyboard, mouse, a monitor, or other computing equipment and peripheral devices. In embodiments, a sample collection room may include a scanner, reader, or other device for reading a label or other code (e.g., a bar-code, a QR-code, cell-phone image, magnetic strip, radiofrequency identification (RFID) code. In embodiments, a sample collection room may include a device for preparing a label, tag, sign, or other identifier, e.g., for affixing to a sample collected in the sample collection room.

In embodiments, a sample collection room may include a refrigerator. In embodiments, a sample collection room may include two refrigerators, or may include a plurality of refrigerators. In embodiments, a refrigerator may be used for sample storage; such a refrigerator may be designated a “BioHazard” refrigerator. In embodiments, a refrigerator may be used to store food, drink, reagents, containers, and consumables for use in sample collection or sample analysis. In embodiments, a refrigerator used for storage of food, drink, reagents, containers, and consumables, may be designated a “Clean” refrigerator.

In embodiments, a sample collection room may include furniture configured for use during collection of a sample from a subject, such as, e.g., a blood sample, a throat swab, a nasal swab, a cheek swab, a sputum sample, a tear sample, or other sample. For example, a sample collection room may include a chair for use by a subject while a sample is collected. In embodiments, such a chair is a reclining chair, having at least two configurations: an upright configuration (in which a subject seated in the chair assumes a normal seated position with the back in a substantially vertical orientation), and a reclined configuration (in which a subject is leaned backwards when seated in the chair). In embodiments, such a reclining chair provides a leg rest, or foot rest, or both, when in the reclined configuration. In embodiments, such a reclining chair does not present a subject with a leg rest, or foot rest, when in the upright configuration.

In embodiments, a sample collection room may include a chair for use by a technician (e.g., a phlebotomist) while collecting a sample from a subject; for use while processing a sample for analysis, storage, or transport; or for use at other times while performing sample collection, processing, analysis, or other tasks. In embodiments, a chair for use by a technician in a sample collection room may be a stool; in embodiments, such a stool may be a low stool configured to aid in positioning a technician adjacent a subject during sample collection. In embodiments, a chair (e.g., a stool) for use by a technician in a sample collection room may have wheels; may have a back; may be adjustable (e.g., the height of the chair seat may be adjustable); and may be colored white or other neutral color.

In embodiments, the results of the analysis are available within a short period of time. In embodiments, a clinician receives results of the analysis within a short period of time after the sample was obtained. In embodiments, the subject receives results of the analysis within a short period of time after the sample was obtained.

In embodiments, the clinical sample may be a small clinical sample. In embodiments, a small clinical sample may be a peripheral blood sample; or a venous blood sample; or an arterial blood sample; or a sample obtained by swab (e.g., a cheek swab, a throat swab, a nasal swab, a vaginal swab, a cervical swab, or other swab sample). In embodiments, the clinical sample may be provided for rapid analysis. In embodiments, a small clinical sample may be provided for rapid analysis. In embodiments, the clinical sample may be provided for analysis at the designated location in a retail store, or for analysis in a location adjacent to, or in close proximity to, the designated location in a retail store; such a clinical sample may be a small clinical sample. In embodiments, the clinical sample may be analyzed at the designated location in a retail store, or in a location adjacent to, or in close proximity to, the designated location in a retail store; such analysis may be rapid analysis.

Applicants disclose methods for obtaining a sample from a subject, comprising providing a sample collection room in a retail store; and collecting a sample from a subject. Applicants disclose methods for obtaining a sample from a subject, comprising providing a sample collection room in a retail store; warming a fingertip, or finger, or hand, or heel, or toe-tip, or toe, of the subject; collecting a sample from a subject; and obtaining the sample. Applicants disclose methods for obtaining a sample from a subject, comprising providing a sample collection room in a retail store, the sample collection room comprising a warming table; warming a fingertip, or finger, or hand, or heel, or toe-tip, or toe, of the subject; and obtaining a sample from the subject. Applicants disclose methods for obtaining a sample from a subject, comprising providing a sample collection room in a retail store, the sample collection room comprising a chair; warming a fingertip, or finger, or hand, or heel, or toe-tip, or toe, of the subject; and obtaining a sample from the subject. Applicants disclose methods for obtaining a sample from a subject, comprising providing a sample collection room in a retail store, the sample collection room comprising a chair having a warming plate or other warming element; warming a fingertip, or finger, or hand, or heel, or toe-tip, or toe, of the subject; and obtaining a sample from the subject. Applicants disclose methods for obtaining a sample from a subject, comprising providing a sample collection room in a retail store, the sample collection room comprising an air-warmer; warming a fingertip, or finger, or hand, or heel, or toe-tip, or toe, of the subject; and obtaining a sample from the subject. Applicants disclose methods for obtaining a sample from a subject, comprising any of the foregoing methods, and providing the subject with a fingertoy, or placing a fingertoy on a finger of a subject following obtaining a sample from the subject. It will be understood that a step of warming a fingertip, or finger, or hand, or heel, or toe-tip, or toe, of a subject may comprise warming by use of a warming table, or warming plate, or air-warmer, or other warming device, or a combination thereof. It will be understood that the foregoing methods may be used together, and may be used in any combination.

Applicant discloses sample collection systems. In embodiments, a sample collection system for obtaining a sample from a subject comprises a sample collection room in a retail store, the sample collection room comprising a warming surface on a chair, table, or counter, and one or more of: a finger warmer; a fingertip warmer; a fingertoy; an air-warmer; a water feature; and a calming feature. In embodiments, a warming surface may comprise one or more of a surface of a warming table, a warming plate, a warming counter, a warming chair, or other furniture for seating comprising a warming element, wherein such a warming surface is configured to adjust one or more of temperature, height, orientation, distance from a subject, and presence or height of a rest configured to support a limb, hand, or foot of a subject. In embodiments, a sample collection system having features as disclosed herein may further comprise a bathroom adjacent said sample collection room; and a pass-through connecting said sample collection room with said bathroom, wherein said pass-through is configured to allow the transfer of a sample from said bathroom to said sample collection room. In embodiments, a sample collection system having features as disclosed herein may further comprise a sample analysis device; or a sample transport container; or both a sample analysis device and a sample transport container. In embodiments, a sample transport container may be configured to hold a plurality of sample containers configured to hold fluid samples having volumes of about 500 μL or less.

Applicant discloses sample analysis systems. In embodiments, a sample collection system for obtaining a sample from a subject comprises a sample analysis system for analyzing a sample from a subject, comprising a sample collection room in a retail store, said sample collection room comprising a sample analysis device configured to analyze a fluid sample having a volume of about 500 μL or less obtained in said sample collection room, a warming surface on a chair, table, or counter, and one or more of: a finger warmer; a fingertip warmer; a fingertoy; an air-warmer; a water feature; and a calming feature. In embodiments, a warming surface may comprise one or more of a surface of a warming table, a warming plate, a warming counter, a warming chair, or other furniture for seating comprising a warming element, wherein such a warming surface is configured to adjust one or more of temperature, height, orientation, distance from a subject, and presence or height of a rest configured to support a limb, hand, or foot of a subject. In embodiments, a sample analysis system having features as disclosed herein may further comprise a bathroom adjacent the sample collection room; and a pass-through connecting the sample collection room with the bathroom, wherein the pass-through is configured to allow the transfer of a sample from said bathroom to said sample collection room.

In embodiments of the sample collection systems and of the sample analysis systems disclosed herein, the sample is a blood or urine sample having a volume selected from the group of volumes of less than about 500 μL, or less than about 250 μL, or less than about 150 μL, or less than about 100 μL, or less than about 50 μL, or less than about 25 μL, or less than about 10 μL, or less than about 5 μL, or less than about 1 μL.

Applicant discloses warming devices. In embodiments, Applicant discloses a warming device having a warming surface, wherein said warming surface comprises one or more of a surface of a warming table, a warming plate, a warming counter, a warming chair, or other furniture for seating comprising a warming element, wherein said warming surface is configured to adjust one or more of temperature, height, orientation, distance from a subject, and presence or height of a rest configured to support a limb, hand, or foot of a subject. In embodiments, a warming device disclosed herein is configured to warm at least a portion of a body of a subject effective to aid in the collection of a blood sample from said subject. In embodiments, the at least a portion of a body of a subject that may be warmed is selected from a fingertip, a finger, and a hand of a subject. In embodiments, a blood sample collected from a subject pursuant to warming at least a portion of the subject's body may have a volume of about 500 μL or less.

Applicant discloses methods for obtaining samples from a subject. In embodiments, a method for obtaining a sample from a subject, said subject having a body, comprises Providing a sample collection system of claim 1 in a retail store, said sample collection system comprising a sample collection room; and Obtaining, in said sample collection room, a sample from said subject. In embodiments, methods for obtaining samples from a subject disclosed herein comprise warming at least a portion of said body of said subject, wherein said warming of at least a portion of the body of the subject comprises warming with a warming surface, wherein said warming surface comprises a surface of a warming table, a warming plate, a warming counter, a warming chair, or other furniture for seating comprising a warming element, wherein said warming surface is configured to adjust one or more of temperature, height, orientation, distance from a subject, and presence or height of a rest configured to support a limb, hand, or foot of a subject. In embodiments, methods for obtaining a sample from a subject may further comprise providing a bathroom adjacent said sample collection room; and providing a pass-through connecting said sample collection room with said bathroom, wherein said pass-through is configured to allow the transfer of a sample from said bathroom to said sample collection room. In embodiments, methods for obtaining a sample from a subject may further comprise one or more of: providing a reclining chair in said sample collection room, said reclining chair being configured for positioning a subject effective for obtaining a sample; providing flowing water in said sample collection room; providing adjustable lighting in the sample collection room; providing a calming image in the sample collection room; providing calming sounds in the sample collection room; and providing a calming scent in the sample collection room. In embodiments of methods for obtaining a sample disclosed herein, the sample is a blood or urine sample having a volume selected from the group of volumes of less than about 500 μL, or less than about 250 μL, or less than 150 μL, or less than about 100 μL, or less than about 50 μL, or less than about 25 μL, or less than about 10 μL, or less than about 5 μL, or less than about 1 μL. In embodiments of methods for obtaining a sample disclosed herein, the sample is analyzed within a short period of time, wherein said short period of time is selected from less than about 5 hours, or less than about 4 hours, or less than about 3 hours, or less than about 2 hours, or less than about 1 hour, or less than about half an hour.

Applicant discloses methods for analyzing samples from a subject. In embodiments, a method for analyzing a sample obtained from a subject, said subject having a body, comprises: providing a sample analyzing system of claim 6 in a retail store, said sample analyzing system comprising a sample collection room; obtaining, in said sample collection room, a sample from said body part of said subject; and analyzing the sample at or adjacent to the retail store. In embodiments, methods for analyzing a sample from a subject may further comprise analyzing the sample at or adjacent to the retail store within a short period of time after obtaining the sample. In embodiments, methods for analyzing a sample from a subject may further comprise transporting the sample to an analysis location; and analyzing the sample at said analysis location within a short period of time after obtaining the sample. In embodiments, methods for analyzing a sample from a subject may further comprise warming at least a portion of said body of said subject, wherein said warming of at least a portion of said body of said subject comprises warming with a warming surface, wherein said warming surface comprises a surface of a warming table, a warming plate, a warming counter, a warming chair, or other furniture for seating comprising a warming element, wherein said warming surface is configured to adjust one or more of temperature, height, orientation, distance from a subject, and presence or height of a rest configured to support a limb, hand, or foot of a subject. In embodiments, methods for analyzing a sample from a subject may further comprise providing a bathroom adjacent said sample collection room; and providing a pass-through connecting said sample collection room with said bathroom, wherein said pass-through is configured to allow the transfer of a sample from said bathroom to said sample collection room. In embodiments, methods for analyzing a sample from a subject may further comprise one or more of the following: providing a warming table in said sample collection room; providing a warming plate in said sample collection room; providing a fingertip warmer in said sample collection room; providing a fingertoy in said sample collection room; providing an air-warmer in said sample collection room; providing a chair or other furniture for seating in said sample collection room, said chair or other furniture for seating comprising a warming plate or other heating element; providing a reclining chair in said sample collection room, said reclining chair being configured for positioning a subject effective for obtaining a sample; providing a reclining chair in said sample collection room, said reclining chair comprising a warming plate or other heating element, said reclining chair being configured for warming at least a portion of a body part of a subject, and for positioning a subject effective for obtaining a sample; providing adjustable lighting in the sample collection room; and providing a calming feature in the sample collection room, wherein said calming feature is selected from the group of calming features consisting of flowing water, a flower, a plant, a calming scent, a calming sound, a calming image, and a calming color. In embodiments, methods for analyzing a sample from a subject may further comprise one or more of providing a reclining chair in said sample collection room, said reclining chair being configured for positioning a subject effective for obtaining a sample; providing flowing water in said sample collection room; providing adjustable lighting in the sample collection room; providing a calming image in the sample collection room; providing calming sounds in the sample collection room; and providing a calming scent in the sample collection room. In embodiments of methods for analyzing a sample disclosed herein, the sample is a blood or urine sample having a volume selected from the group of volumes of less than about 500 μL, or less than about 250 μL, or less than 150 μL, or less than about 100 μL, or less than about 50 μL, or less than about 25 μL, or less than about 10 μL, or less than about 5 μL, or less than about 1 μL. In embodiments of methods for analyzing a sample disclosed herein, the sample is analyzed within a short period of time, wherein said short period of time is selected from less than about 5 hours, or less than about 4 hours, or less than about 3 hours, or less than about 2 hours, or less than about 1 hour, or less than about half an hour.

In embodiments, the sample is a small sample. For example, in embodiments, the sample is a small volume sample of blood, or of urine, or of saliva, or of tears, or of stool, or other bodily secretion or excretion. In embodiments of the methods for obtaining a sample from a subject, the sample is obtained from a finger-stick. In embodiments, the sample is a small sample obtained from a small lancet puncture in the skin of the subject (a finger-stick). For example, in embodiments, the sample comprises a few drops, or two drops, or one drop, of blood obtained from a finger-stick. For example, in embodiments, the sample is a small sample having a volume of less than about 200 μL, or less than about 150 μL, or less than about 100 μL, or less than about 75 μL, or less than about 50 μL, or less than about 25 μL, or less.

Applicants disclose methods for analyzing a sample obtained from a subject, comprising providing a sample collection room in a retail store; collecting a sample from a subject; and analyzing the sample. Applicants disclose methods for analyzing a sample obtained from a subject, comprising providing a sample collection room in a retail store; warming a fingertip, or finger, or hand, or heel, or toe-tip, or toe, of the subject; collecting a sample from a subject; and analyzing the sample. Applicants disclose methods for analyzing a sample obtained from a subject, comprising providing a sample collection room in a retail store, the sample collection room comprising a warming table; warming a fingertip, or finger, or hand, or heel, or toe-tip, or toe, of the subject; collecting a sample from a subject; and analyzing the sample. Applicants disclose methods for analyzing a sample obtained from a subject, comprising providing a sample collection room in a retail store, the sample collection room comprising a chair; warming a fingertip, or finger, or hand, or heel, or toe-tip, or toe, of the subject; collecting a sample from a subject; and analyzing the sample. Applicants disclose methods for analyzing a sample obtained from a subject, comprising providing a sample collection room in a retail store, the sample collection room comprising a chair having a warming plate or other warming element; warming a fingertip, or finger, or hand, or heel, or toe-tip, or toe, of the subject; collecting a sample from a subject; and analyzing the sample. Applicants disclose methods for analyzing a sample obtained from a subject, comprising providing a sample collection room in a retail store, the sample collection room comprising an air-warmer; warming a fingertip, or finger, or hand, or heel, or toe-tip, or toe, of the subject; collecting a sample from a subject; and analyzing the sample. Applicants disclose methods for analyzing a sample obtained from a subject, comprising any of the foregoing methods, and providing the subject with a fingertoy, or placing a fingertoy on a finger of a subject following obtaining a sample from the subject. It will be understood that a step of warming a fingertip, or finger, or hand, or heel, or toe-tip, or toe, of a subject may comprise warming by use of a warming table, or warming plate, or air-warmer, or other warming device, or a combination thereof. It will be understood that the foregoing methods may be used together, and may be used in any combination.

In embodiments, the sample is analyzed at, or adjacent to, or near to, the sample collection room in the retail store. In embodiments, the sample is analyzed at a location other than at the retail store. In embodiments of the methods for analyzing a sample obtained from a subject, the sample is a small sample. For example, in embodiments, the sample is obtained from a finger-stick. For example, in embodiments, the sample comprises a few drops, or two drops, or one drop, of blood obtained from a small lancet puncture in the skin of the subject. For example, in embodiments, the sample is a small volume sample of blood, or of urine, or of saliva, or of tears, or other bodily secretion or excretion. For example, in embodiments, the sample is a small sample having a volume of less than about 200 μL, or less than about 150 μL, or less than about 100 μL, or less than about 75 μL, or less than about 50 μL, or less than about 25 μL, or less.

Accordingly, Applicants disclose methods for obtaining and analyzing a sample obtained from a subject, comprising: providing a sample collection room in a retail store; collecting a sample from a subject in the sample collection room; and analyzing the sample. Applicants disclose methods for obtaining and analyzing a sample obtained from a subject, comprising: providing a sample collection room in a retail store; collecting a small sample from a subject in the sample collection room; and analyzing the small sample. Applicants disclose methods for obtaining and analyzing a sample obtained from a subject, comprising: providing a sample collection room in a retail store; collecting a sample from a subject in the sample collection room; and analyzing the sample at the retail store location, or adjacent to the retail store location.

In embodiments, a sample collection room may include a comfortable chair or seating location for a subject, e.g., for use by a subject during collection of a sample from the subject. In embodiments, a comfortable chair may be a reclining chair, having an upright configuration and a reclined configuration. In embodiments, a comfortable chair may be a reclining chair having an upright configuration and a plurality of reclined configurations. In embodiments, a sample collection room may include a refrigerator, a cabinet, a sink, storage units, a work surface (which may be configured for sample collection, for writing, or for other purposes), and other furniture and architectural elements. In embodiments, a sample collection room may include a centrifuge. In embodiments, a sample collection room may include one or more calming features, such as, e.g., a calming image (including still images and video images); a sound system for providing calming sounds and music; lighting (e.g., soft, white lighting) configured to calm a subject; flowing water; plants, including flowers; and other calming features as disclosed herein.

In embodiments, a sample collection room as disclosed herein may include a sample analysis device or system; such a sample analysis device or system may be an automated sample analysis device or system, or may be a semi-automated sample analysis device or system. In embodiments, a sample collection room may include a computer, computer accessories, communication equipment and capability effective to link a computer, cell phone, or other device with WiFi, internet, cable, or other connections. In embodiments, a sample collection room may include a scanner, a label printer, and other automated reading and printing equipment.

Accordingly, Applicants disclose herein systems comprising a room in a retail location, the room being configured for obtaining a clinical sample such as a blood sample. In embodiments, such systems comprise a room in a retail location, the room being configured for obtaining a clinical sample such as a blood sample, the room including one or more of a warming table, a calming feature, a chair, a pass-through to an adjacent bathroom, adjustable lighting, calming scents, and a sound source providing calming sounds. In embodiments, the chair is a reclining chair. In embodiments, the system includes an adjacent bathroom.

In embodiments, the sample collection room may be part of a sample collection center, or wellness center, including a waiting room and a sample collection room. In embodiments, a sample collection room, or wellness center, may include a waiting room, a sample collection room, and a bathroom. In embodiments, a bathroom that is part of a sample collection room, or wellness center, may include a sink (e.g., for handwashing) and a toilet. In embodiments, a bathroom and a sample collection room may be connected via a passageway configured for passing a sample from the bathroom to the sample collection room.

Accordingly, Applicants disclose embodiments of systems for obtaining samples, and systems for analyzing samples, including at least the following.

A room in a retail location configured for obtaining a blood sample including warming table, a calming feature, and a chair, the room being configured for obtaining a clinical sample such as a blood sample. In embodiments, the chair is a reclining chair.

A room in a retail location configured for obtaining a blood sample including a warming table, a calming feature, a pass-through to an adjacent bathroom, and a reclining chair.

A room in a retail location configured for obtaining a blood sample including a warming table, a calming feature, adjustable lighting, a pass-through to an adjacent bathroom, and a reclining chair.

A room in a retail location configured for obtaining a blood sample including a warming table, a calming feature, adjustable lighting, calming scents, a pass-through to an adjacent bathroom, and a reclining chair.

A room in a retail location configured for obtaining a blood sample including a warming table, a calming feature, a calming image, calming sounds, adjustable lighting, calming scents, a pass-through to an adjacent bathroom, and a reclining chair.

A room in a retail location configured for obtaining a blood sample including a warming table, a calming feature, a calming image, calming sounds, calming colors, adjustable lighting, calming scents, a pass-through to an adjacent bathroom, and a reclining chair.

Applicants disclose methods for obtaining samples, and for analyzing samples, including the following methods.

A method for obtaining a sample from a subject, said subject having a body part, comprising: providing a sample collection room in a retail store; and obtaining a sample from said body part of said subject, wherein said obtaining is performed in said sample collection room.

A method for analyzing a sample obtained from a subject, said subject having a body part, comprising: providing a sample collection room in a retail store; obtaining, in said sample collection room, a sample from said body part of said subject; and analyzing the sample at or adjacent to the retail store.

A method for analyzing a sample obtained from a subject, said subject having a body part, comprising: providing a sample collection room in a retail store; obtaining, in said sample collection room, a sample from said body part of said subject; and analyzing the sample at or adjacent to the retail store within a short period of time after obtaining the sample.

A method for analyzing a sample obtained from a subject, said subject having a body part, comprising: providing a sample collection room in a retail store; obtaining, in said sample collection room, a sample from said body part of said subject; transporting the sample to an analysis location; and analyzing the sample at said analysis location within a short period of time after obtaining the sample. In embodiments, obtaining said sample comprises warming the body part of the subject. In embodiments, warming the body part comprises warming with a warming table. In embodiments, warming the body part comprises warming with a warming plate. In embodiments, warming the body part comprises warming with an air-warmer. In embodiments, the body part comprises a fingertip, a finger, a plurality of fingers, a hand, or other body part or body parts.

In embodiments of the foregoing methods, the methods may comprise warming a body part; for example, a body part may be warmed prior to obtaining a sample from the body part. In embodiments, warming of the body part comprises warming with a warming table. In embodiments, warming of the body part comprises warming with a warming plate. In embodiments, warming of the body part comprises warming with a finger warmer. In embodiments, warming of the body part comprises warming with a fingertip warmer. In embodiments, warming of the body part comprises warming with an air-warmer. In embodiments, the body part comprises a fingertip, a finger, a plurality of fingers, a hand, or other body part or body parts.

In embodiments, methods for analyzing a sample comprise analyzing said sample for the presence of an analyte. In embodiments, methods for analyzing a sample comprise analyzing said sample for the presence of a plurality of analytes. In embodiments, methods for analyzing a sample comprise analyzing said sample for the presence of a plurality of analytes, wherein said plurality of analytes includes two or more of a nucleic acid analyte, a peptide (or polypeptide) analyte, an inorganic analyte, a cell or cell type, and other analytes. In embodiments, methods for analyzing a sample comprise analyzing said sample for the presence of a plurality of analytes, wherein said plurality of analytes are detected by methods including two or more of nucleic acid hybridization methods, antibody binding methods, general chemistry methods, cytometric methods, ion-selective electrode methods, and other methods.

In embodiments, methods for analyzing a sample comprise analyzing a sample by automated analysis devices, or by semi-automated analysis devices, or systems comprising such devices. In embodiments, methods for analyzing a sample comprise analyzing a sample at the sample collection center, e.g., in the sample collection room, or in a room adjacent to the sample collection room, or in a room near to the sample collection room. In embodiments, methods for analyzing a sample comprise analyzing the sample in a short period of time. In embodiments, methods for analyzing a sample comprise analyzing a small volume sample. In embodiments, methods for analyzing a sample comprise analyzing a small volume sample in a short period of time.

In embodiments, methods for collecting a sample comprise centrifuging a sample after collection; in embodiments, the sample comprises a small volume sample. In embodiments, methods for collecting a sample comprise centrifuging a sample within a short period of time after sample collection; in embodiments, the sample comprises a small volume sample. In embodiments, methods for analyzing a sample comprise centrifuging a sample; in embodiments, the sample comprises a small volume sample. In embodiments, methods for analyzing a sample comprise centrifuging and analyzing a small volume sample within a short period of time following collection of the sample.

In embodiments, these methods may further comprise providing a bathroom adjacent said sample collection room; and providing a pass-through connecting said sample collection room with said bathroom, wherein said pass-through is configured to allow the transfer of a sample from said bathroom to said sample collection room. In embodiments, the sample comprises a urine sample. In embodiments, the sample comprises a stool sample. In embodiments, the sample comprises a sample obtained using a swab. In embodiments, a sample obtained using a swab may comprise a throat swab, a cheek swab, a nasal swab, a vaginal swab, or other swab.

In embodiments, these methods may further comprise one or more of: providing a reclining chair in said sample collection room, said reclining chair being configured for positioning a subject effective for obtaining a sample; providing a reclining chair in said sample collection room, said reclining chair having a warming plate or other warming element and being configured for positioning a subject effective for obtaining a sample; providing a warming table in said sample collection room, said warming table being effective to warm a body part (e.g., a fingertip, finger, fingers, or hand); providing a warming plate in said sample collection room, said warming plate being effective to warm a body part (e.g., a fingertip, finger, fingers, or hand); providing an air-warmer in said sample collection room, said air-warmer being effective to warm a body part (e.g., a fingertip, finger, fingers, or hand); and providing a calming feature in the sample collection room. In embodiments, the calming feature may be selected from the group of calming features consisting of flowing water, a flower, a plant, a calming scent, a calming sound, a calming image, and a calming color.

In embodiments, these methods may further comprise providing a reclining chair in said sample collection room, said reclining chair being configured for positioning a subject effective for obtaining a sample; providing flowing water in said sample collection room; providing adjustable lighting in the sample collection room; providing calming sounds in the sample collection room; and providing a calming scent in the sample collection room. In embodiments, these methods may further comprise providing a calming image in the sample collection room.

In embodiments, these methods may further comprise providing a movable chair or stool suitable for use by a technician during sample collection; may further comprise providing a refrigerator, or refrigerators, for use before, during, or after sample collection; may further comprise providing a bar-code or label reader; may further comprise providing a bar-code or label printer; may further comprise providing a computer, and may include providing computer peripheral devices; may include providing storage space; and may include providing other furniture, devices, materials, and implements.

Applicants further provide systems, including the following systems.

A system for obtaining a sample from a subject, comprising a sample collection room in a retail store, said sample collection room comprising a warming table. A system for obtaining a sample from a subject, comprising a sample collection room in a retail store, said sample collection room comprising a warming plate. A system for obtaining a sample from a subject, comprising a sample collection room in a retail store, said sample collection room comprising a chair, couch, bench, or other piece of furniture for seating, wherein the chair, couch, bench, or other piece of furniture has a warming plate or warming element in an armrest of the chair, couch, bench, or other piece of furniture. A system for obtaining a sample from a subject, comprising a sample collection room in a retail store, said sample collection room comprising a finger warmer. A system for obtaining a sample from a subject, comprising a sample collection room in a retail store, said sample collection room comprising a fingertip warmer. A system for obtaining a sample from a subject, comprising a sample collection room in a retail store, said sample collection room comprising an air-warmer.

A system for obtaining a sample from a subject, comprising a sample collection room in a retail store, said sample collection room comprising a warming table; a reclining chair (or other piece of furniture for seating) configured for positioning a subject effective for obtaining a sample; and a calming feature. In embodiments, a warming table may be configured to adjust one or more of temperature, height, longitudinal orientation, lateral orientation, distance from a subject, and presence or height of a rest. In embodiments, a chair (or other piece of furniture for seating) may include a warming plate or warming element in an armrest. In embodiments, a calming feature may be selected from the group of calming features consisting of flowing water, a flower, a plant, a calming scent, a calming sound, a calming image, and a calming color.

In embodiments, a system for obtaining a sample from a subject comprises a sample collection room in a retail store, said sample collection room comprising a sample transport container configured to hold a plurality of sample containers, said sample containers configured to hold fluid samples having volumes of about 500 μL or less, and one or more of: a warming table, a warming plate, a chair or other furniture for seating comprising a warming plate, a finger warmer, a fingertip warmer, and an air-warmer. In embodiments, a system for analyzing a sample from a subject comprises a sample collection room in a retail store, said sample collection room comprising a sample analysis device configured to analyze a fluid sample having volumes of about 500 μL or less obtained in said sample collection room, and one or more of: a warming table; a reclining chair configured for positioning a subject effective for obtaining a sample; and a calming feature. In embodiments, a system for analyzing a sample from a subject comprises a sample collection room in a retail store, said sample collection room comprising a sample analysis device configured to analyze a fluid sample having volumes of about 500 μL or less obtained in said sample collection room, and one or more of: a chair or other furniture for seating, said chair or other furniture for seating comprising a warming plate or other heating element, the chair or other furniture for seating being configured to warm a body part of said subject, and being configured for positioning a subject effective for obtaining a sample; and a calming feature. In embodiments, a warming table of such a system is configured to adjust one or more of temperature, height, orientation, distance from a subject, and presence or height of a rest. In embodiments, a calming feature of such a system is selected from the group of calming features consisting of flowing water, a flower, a plant, a calming scent, a calming sound, a calming image, and a calming color. In embodiments, a system further comprises a bathroom adjacent said sample collection room; and a pass-through connecting said sample collection room with said bathroom, wherein said pass-through is configured to allow the transfer of a sample from said bathroom to said sample collection room. In embodiments, a system further comprises one or more of a fingertoy; flowing water in said sample collection room; adjustable lighting in said sample collection room; a calming image; a wall or wall hanging having a calming color; a sound system configured for providing calming sounds in the sample collection room; and a scent source configured to provide a calming scent in the sample collection room, wherein said scent source is selected from a flower, a candle, an oil, a scented wick, and an aerosol.

In embodiments, the system may further comprise a bathroom adjacent said sample collection room; and a pass-through connecting said sample collection room with said bathroom, wherein said pass-through is configured to allow the transfer of a sample from said bathroom to said sample collection room. In embodiments, the sample comprises a urine sample.

In embodiments, such systems may further comprise one or more of: flowing water in said sample collection room; adjustable lighting in said sample collection room; a calming image; a wall or wall hanging having a calming color; a sound system configured for providing calming sounds in the sample collection room; and a scent source configured to provide a calming scent in the sample collection room. In embodiments, a scent source may be selected from a flower, a candle, an oil, a scented wick, and an aerosol.

In embodiments, systems as disclosed herein may comprise a movable chair or stool suitable for use by a technician during sample collection; may comprise a refrigerator, or refrigerators, for use before, during, or after sample collection; may comprise a bar-code or label reader; may comprise a bar-code or label printer; may comprise a computer, and may include computer peripheral devices; may include storage space; and may include other furniture, devices, materials, and implements.

In embodiments, systems as disclosed herein may further include a warming plate, or a finger warmer, or a fingertip warmer, or an air-warmer, or combination thereof. In embodiments, systems as disclosed herein may further include a fingertoy. It will be understood that systems as disclosed herein may include combinations of some or all of the elements disclosed herein.

In embodiments, systems disclosed herein are suitable for use with a blood, urine, stool, or other sample having a volume selected from the group of volumes of less than about 250 μL, or less than 150 μL, or less than about 100 μL, or less than about 50 μL, or less than about 25 μL, or less than about 10 μL, or less than about 5 μL, or less than about 1 μL.

Applicants disclose herein a sample collection room in a retail store, said sample collection room comprising one or more of: a warming table, a warming plate, a chair or other furniture for seating comprising a warming plate, a finger warmer, a fingertip warmer, and an air-warmer. In embodiments, a sample collection room in a retail store further comprises a sample analysis device, a sample transport container configured to hold a plurality of sample containers, or both. In embodiments, a sample collection room in a retail store includes a sample analysis device or sample analysis system. In embodiments, a sample collection room in a retail store is located adjacent to a sample analysis device or sample analysis system. In embodiments, such a sample analysis device is configured to analyze fluid samples having volumes of about 500 μL or less. In embodiments, a sample collection room in a retail store includes sample containers configured to hold fluid samples having volumes of about 500 μL or less. In embodiments, a sample collection room in a retail store is adjacent one or more of a waiting room and a bathroom. In embodiments, a sample collection room in a retail store is adjacent a bathroom, and further comprises a pass-through connecting said bathroom with said sample collection room.

Applicants disclose herein a warming device selected from a warming table, a warming plate, a chair or other furniture for seating comprising a warming plate, a finger warmer, a fingertip warmer, and an air-warmer. In embodiments, a warming device is configured to warm a body part of a subject effective to aid in the collection of a blood sample from said body part. In embodiments, said body part is selected from a fingertip, a finger, and a hand. In embodiments, a warming device is effective to aid in the collection of a blood sample having a volume of about 500 μL or less.

Applicants disclose herein a method for obtaining a sample from a subject, said subject having a body part, comprising: providing a sample collection room in a retail store; and obtaining, in said sample collection room, a sample from said body part of said subject. Applicants disclose herein a further method for analyzing a sample obtained from a subject, said subject having a body part, comprising: providing a sample collection room in a retail store; obtaining, in said sample collection room, a sample from said body part of said subject; and analyzing the sample at or adjacent to the retail store. Applicants disclose herein a further method for analyzing a sample obtained from a subject, said subject having a body part, comprising: providing a sample collection room in a retail store; obtaining, in said sample collection room, a sample from said body part of said subject; and analyzing the sample at or adjacent to the retail store within a short period of time after obtaining the sample. Applicants disclose herein a further method for analyzing a sample obtained from a subject, said subject having a body part, comprising: providing a sample collection room in a retail store; obtaining, in said sample collection room, a sample from said body part of said subject; transporting the sample to an analysis location; and analyzing the sample at said analysis location within a short period of time after obtaining the sample. In embodiments of such methods, obtaining said sample comprises warming said body part of said subject. In embodiments, warming a body part comprises warming with a warming table; a warming plate; a finger warmer; a fingertip warmer; an air-warmer; furniture for seating comprising a warming plate or other heating element; or a combination thereof. In embodiments, the methods further comprise: providing a bathroom adjacent said sample collection room; and providing a pass-through connecting said sample collection room with said bathroom, wherein said pass-through is configured to allow the transfer of a sample from said bathroom to said sample collection room. In embodiments, the methods further comprise one or more of the following: providing a warming table in said sample collection room; providing a warming plate in said sample collection room; providing a fingertip warmer in said sample collection room; providing a fingertoy in said sample collection room; providing an air-warmer in said sample collection room; providing a chair or other furniture for seating in said sample collection room, said chair or other furniture for seating comprising a warming plate or other heating element; providing a reclining chair in said sample collection room, said reclining chair being configured for positioning a subject effective for obtaining a sample; providing a reclining chair in said sample collection room, said reclining chair comprising a warming plate or other heating element, said reclining chair being configured for warming at least a portion of a body part of a subject, and for positioning a subject effective for obtaining a sample; providing adjustable lighting in the sample collection room; and providing a calming feature in the sample collection room, wherein said calming feature is selected from the group of calming features consisting of flowing water, a flower, a plant, a calming scent, a calming sound, a calming image, and a calming color.

In embodiments of the methods disclosed herein, a sample has a volume selected from the group of volumes of less than about 500 μL, or less than about 250 μL, or less than 150 μL, or less than about 100 μL, or less than about 50 μL, or less than about 25 μL, or less than about 10 μL, or less than about 5 μL, or less than about 1 μL. In embodiments of the methods disclosed herein, a sample is analyzed within a short period of time, wherein said short period of time is selected from less than about 5 hours, or less than about 4 hours, or less than about 3 hours, or less than about 2 hours, or less than about 1 hour, or less than about half an hour. In embodiments of the methods disclosed herein, a sample is analyzed within a short period of time after obtaining the sample, wherein the short period of time is selected from less than about 5 hours, or less than about 4 hours, or less than about 3 hours, or less than about 2 hours, or less than about 1 hour, or less than about half an hour.

The methods, rooms, and systems disclosed herein are suited for providing subjects with pleasant surroundings, and convenient access to locations where a sample may be obtained for analysis. The methods, rooms, and systems disclosed herein a provide methods and means for obtaining samples from subjects in a pleasant and convenient manner. The methods, rooms, and systems disclosed herein are configured to reduce discomfort to a subject during sample collection; to reduce anxiety in a subject related to sample collection; and thus are useful in improving subject compliance with diagnostic testing, and in improving subject engagement with, desire to participate in, an enjoyment of, diagnostic testing. Greater subject participation and greater subject compliance with diagnostic testing is believed to improve clinical outcomes, for subjects suspected of suffering from a disease or medical conditions, and for healthy subjects who may thus obtain more and more comprehensive baseline clinical data for subsequent comparison and use. Increased convenience of diagnostic testing provided by the methods, rooms, and systems disclosed herein provide decreased cost, increased availability, greater subject compliance, and greater subject engagement with diagnostic testing which is believed to lead to improved health and clinical outcomes. Thus, the methods, rooms, and systems disclosed herein provide advantages to subjects who wish to provide a sample (e.g., a clinical sample for medical analysis or diagnosis) and provide advantages over the art.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1A shows a plan view of a sample collection room within a retail location, having a waiting room, a sample collection room, and a bathroom. Exemplary positioning of furniture, work surfaces, and doors are indicated in the figure.

FIG. 1B shows a plan view of a sample collection room within a retail location, having a waiting room, a sample collection room, a centrifuge, and a bathroom. Exemplary positioning of furniture, work surfaces, and doors are indicated in the figure.

FIG. 1C shows a plan view of a sample collection room within a retail location, having a waiting room, a sample collection room, a transport container, and a bathroom. Exemplary positioning of furniture, work surfaces, and doors are indicated in the figure.

FIG. 1D shows a plan view of a sample collection room within a retail location, having a waiting room, a sample collection room, a centrifuge, a refrigerator, a transport container, and a bathroom. Exemplary positioning of furniture, work surfaces, and doors are indicated in the figure.

FIG. 2 shows an example of a warming table having features as disclosed herein.

FIG. 3 shows an example of a warming table having features as disclosed herein, including an arm rest.

FIG. 4A shows an example of a warming plate having features as disclosed herein, providing a top view of a warming plate, illustrating placement of fingers onto the plate for use in warming fingers of a hand of a subject.

FIG. 4B shows an example of a warming plate having features as disclosed herein, providing a perspective view of a warming plate in use warming fingers of a hand of a subject.

FIG. 4C shows an example of a warming plate having features as disclosed herein, providing a front perspective view of a chair including a warming plate in an arm rest, and of a cabinet attached to the chair, the cabinet including a warming plate on its top surface.

FIG. 5A shows an example of a finger warmer having features as disclosed herein, providing a schematic top view of a finger warmer in an extended (unrolled) configuration.

FIG. 5B shows an example of a finger warmer having features as disclosed herein, providing a front (right) and back (left) view of a finger warmer.

FIG. 5C shows a finger warmer wrapped around a finger of a subject.

FIG. 6A shows an example of a fingertip warmer having features as disclosed herein, providing a side perspective view of a fingertip warmer.

FIG. 6B shows an example of a fingertip warmer having features as disclosed herein, providing an image of a fingertip warmer in place around the fingertip of a subject.

FIG. 7 shows an example of an air-warmer having features as disclosed herein.

FIG. 8 shows an example of a fingertip cover toy for placement over a child's finger prior to, or after obtaining a blood sample from the finger.

### DETAILED DESCRIPTION

Methods, devices, and systems for obtaining a biological sample from a subject, such as, e.g., blood, are disclosed, for example, in U.S. Patent Application 61/875,092, filed Sep. 8, 2013; in U.S. Patent Application 61/894,166, filed Oct. 22, 2013; in U.S. Patent Application 61/852,484, filed Mar. 15, 2013; in U.S. patent application Ser. No. 14/214,854, filed Mar. 15, 2014; in U.S. Patent Application 61/803,449, filed Mar. 19, 2013; and in U.S. patent application Ser. No. 14/220,013, filed Mar. 19, 2014, the disclosures of all of which patent applications are hereby incorporated by reference in their entireties.

Description and disclosure of examples of obtaining samples and of sample analysis methods, devices, and systems, including automated analysis devices, semi-automated analysis devices, and systems comprising such devices, which may be used with the methods and systems disclosed herein may be found, for example, in: U.S. Pat. No. 8,088,593; U.S. Pat. No. 8,380,541; U.S. Pat. No. 8,435,738; U.S. Pat. No. 8,475,739; U.S. patent application Ser. No. 13/769,817, filed Feb. 18, 2013; U.S. patent application Ser. No. 13/769,818, filed Feb. 18, 2013; U.S. patent application Ser. No. 13/769,820, filed Feb. 18, 2013; U.S. patent application Ser. No. 14/183,503, filed Feb. 18, 2014; U.S. patent application Ser. No. 13/769,798, filed Feb. 18, 2013; U.S. patent application Ser. No. 13/769,779, filed Feb. 18, 2013; U.S. patent application Ser. No. 13/769,820, filed Feb. 18, 2013; PCT/US2012/57155, filed Sep. 25, 2012; U.S. patent application Ser. No. 13/244,949, filed Sep. 26, 2011; U.S. Application Ser. No. 61/800,606, filed Mar. 15, 2013; U.S. application Ser. No. 14/214,850, filed Mar. 15, 2014; U.S. Application Ser. No. 61/766,095, filed Feb. 18, 2013; International Application Serial No. PCT/US2014/016593, filed Feb. 14, 2014; U.S. Application Ser. No. 61/673,245, filed Jul. 18, 2012; U.S. application Ser. No. 13/945,202, filed Jul. 18, 2013; U.S. Patent Application 61/805,923, filed Mar. 27, 2013; International Application Serial No. PCT/US2014/016697, filed Feb. 18, 2014; U.S. Patent Application 61/803,449, filed Mar. 19, 2013; U.S. Patent Application 61/815,030, filed Sep. 7, 2013; International Patent Application PCT/US2013/052141, filed Jul. 25, 2013; U.S. Patent Application Ser. No. 61/879,664, filed Sep. 18, 2013; U.S. application Ser. No. 14/479,190, filed Sep. 5, 2014; U.S. Patent Application Ser. No. 61/837,167, filed Jun. 19, 2013; U.S. application Ser. No. 14/309,689, filed Jun. 19, 2014; U.S. Patent Application Ser. No. 61/874,976, filed Sep. 6, 2013; U.S. application Ser. No. 14/479,241, filed Sep. 5, 2014; U.S. application Ser. No. 14/479,245, filed Sep. 5, 2014; U.S. Patent Application Ser. No. 61/766,116, filed Feb. 18, 2013; U.S. application Ser. No. 13/951,063, filed Jul. 25, 2013; U.S. Patent Application Ser. No. 61/802,194, filed Mar. 15, 2013; U.S. application Ser. No. 13/951,449, filed Jul. 25, 2013; U.S. patent application Ser. No. 13/244,947 filed Sep. 26, 2011; and U.S. application Ser. No. 13/244,946, filed Sep. 26, 2011; the disclosures of all which patents and patent applications are hereby incorporated by reference in their entireties.

Systems, devices, and methods disclosed herein provide without limitation sample collection rooms, sample collection methods, and related devices, apparatus, and methods. Sample collection rooms, systems, devices, and methods may include sample analysis devices or sample analysis systems. As used herein, a sample analysis system may include a sample analysis device, and the term “sample analysis device” may also refer to systems for sample analysis that include such devices. In embodiments, a sample analysis device may be an automated sample analysis device; or may be a semi-automated sample analysis device.

In embodiments, for example, a sample analysis device may be useful for receiving a sample. A sample analysis device may be useful for preparing, or for processing a sample. A sample analysis device may be useful for performing an assay on a sample. A device may be useful for obtaining data from a sample. A sample analysis device may be useful for transmitting data obtained from a sample. A sample analysis device may be useful for disposing of a sample following processing or assaying of a sample.

A sample analysis device may be part of a system, a component of which may be an automated sample analysis device, or a semi-automated sample analysis device. A sample analysis device may be configured to facilitate collection of a sample, prepare a sample for a clinical test, or effect a chemical reaction with one or more reagents or other chemical or physical processing, as disclosed herein. A sample analysis device may be configured to obtain data from a sample. A sample analysis device may be configured to transmit data obtained from a sample. A sample analysis device may be configured to analyze data from a sample. An automated sample analysis device may be configured to communicate with another device, or a laboratory, or an individual affiliated with a laboratory, to analyze data obtained from a sample.

A sample analysis device may include, or may be, an automated sample analysis device. A sample analysis device may be configured to perform processing steps or actions on a sample obtained from a subject. Sample processing may include sample preparation, including, e.g., sample dilution, division of a sample into aliquots, extraction, contact with a reagent, filtration, separation, centrifugation, or other preparatory or processing action or step. An automated sample analysis device may be configured to perform one or more sample preparation action or step on the sample. Optionally, a sample may be prepared for a chemical reaction and/or physical processing step. A sample preparation action or step may include one or more of the following: centrifugation, separation, filtration, dilution, enriching, purification, precipitation, incubation, pipetting, transport, chromatography, cell lysis, cytometry, pulverization, grinding, activation, ultrasonication, micro column processing, processing with magnetic beads, processing with nanoparticles, or other sample preparation action or steps. For example, sample preparation may include one or more step to separate blood into serum and/or particulate fractions, or to separate any other sample into various components. Sample preparation may include one or more step to dilute and/or concentrate a sample, such as a blood sample, or other biological samples. Sample preparation may include adding an anti-coagulant or other ingredients to a sample. Sample preparation may also include purification of a sample. In embodiments, all sample processing, preparation, or assay actions or steps are performed by a single device. In embodiments, all sample processing, preparation, or assay actions or steps are performed within a housing of a single device. In embodiments, most sample processing, preparation, or assay actions or steps are performed by a single device, and may be performed within a housing of a single device. In embodiments, many sample processing, preparation, or assay actions or steps are performed by a single device, and may be performed within a housing of a single device. In embodiments, sample processing, preparation, or assay actions or steps may be performed by more than one device.

A sample analysis device may be configured to run one or more assay on a sample, and to obtain data from the sample. An assay may include one or more physical or chemical treatments, and may include running one or more chemical or physical reactions. A sample analysis device may be configured to perform one, two or more assays on a small sample of bodily fluid. One or more chemical reaction may take place on a sample having a volume, as described elsewhere herein. For example one or more chemical reaction may take place in a pill having less than femtoliter volumes. In an instance, the sample collection unit is configured to receive a volume of the bodily fluid sample equivalent to a single drop or less of blood or interstitial fluid. In embodiments, the volume of a sample may be a small volume, where a small volume may be a volume that is less than about 1000 μL, or less than about 500 μL, or less than about 250 μL, or less than about 150 μL, or less than about 100 μL, or less than about 75 μL, or less than about 50 μL, or less than about 40 μL, or less than about 20 μL, or less than about 10 μL, or other small volume. In embodiments, all sample assay actions or steps are performed on a single sample. In embodiments, all sample assay actions or steps are performed by a single device. In embodiments, all sample assay actions or steps are performed within a housing of a single device. In embodiments, most sample assay actions or steps are performed by a single device, and may be performed within a housing of a single device. In embodiments, many sample assay actions or steps are performed by a single device, and may be performed within a housing of a single device. In embodiments, sample processing, preparation, or assay actions or steps may be performed by more than one device.

A sample analysis device may be configured to perform a plurality of assays on a sample. In embodiments, an automated sample analysis device may be configured to perform a plurality of assays on a single sample. In embodiments, an automated sample analysis device may be configured to perform a plurality of assays on a single sample, where the sample is a small sample. For example, a small sample may have a sample volume that is a small volume of less than about 1000 μL, or less than about 500 μL, or less than about 250 μL, or less than about 150 μL, or less than about 100 μL, or less than about 75 μL, or less than about 50 μL, or less than about 40 μL, or less than about 20 μL, or less than about 10 μL, or other small volume. A sample analysis device may be capable of performing multiplexed assays on a single sample. A plurality of assays may be run simultaneously; may be run sequentially; or some assays may be run simultaneously while others are run sequentially. One or more control assays and/or calibrators (e.g., including a configuration with a control of a calibrator for the assay/tests) can also be incorporated into the device; control assays and assay on calibrators may be performed simultaneously with assays performed on a sample, or may be performed before or after assays performed on a sample, or any combination thereof. In embodiments, all sample assay actions or steps are performed by a single device. In embodiments, all of a plurality of assay actions or steps are performed within a housing of a single device. In embodiments, most sample assay actions or steps, of a plurality of assays, are performed by a single device, and may be performed within a housing of a single device. In embodiments, many sample assay actions or steps, of a plurality of assays, are performed by a single device, and may be performed within a housing of a single device. In embodiments, sample processing, preparation, or assay actions or steps may be performed by more than one device.

In embodiments, all of a plurality of assays may be performed in a short time period. In embodiments, such a short time period comprises less than about three hours, or less than about two hours, or less than about one hour, or less than about 40 minutes, or less than about 30 minutes, or less than about 25 minutes, or less than about 20 minutes, or less than about 15 minutes, or less than about 10 minutes, or less than about 5 minutes, or less than about 4 minutes, or less than about 3 minutes, or less than about 2 minutes, or less than about 1 minute, or other short time period.

A sample analysis device may be configured to detect one or more signals relating to the sample. A sample analysis device may be configured to identify one or more properties of the sample. For instance, the sample analysis device may be configured to detect the presence or concentration of one analyte or a plurality of analytes or a disease condition in the sample (e.g., in or through a bodily fluid, secretion, tissue, or other sample). Alternatively, the sample analysis device may be configured to detect a signal or signals that may be analyzed to detect the presence or concentration of one or more analytes (which may be indicative of a disease condition) or a disease condition in the sample. The signals may be analyzed on board the device, or at another location. Running a clinical test may or may not include any analysis or comparison of data collected.

A chemical reaction or other processing step may be performed, with or without the sample. Examples of steps, tests, or assays that may be prepared or run by the device may include, but are not limited to immunoassay, nucleic acid assay, receptor-based assay, cytometric assay, colorimetric assay, enzymatic assay, electrophoretic assay, electrochemical assay, spectroscopic assay, chromatographic assay, microscopic assay, topographic assay, calorimetric assay, turbidmetric assay, agglutination assay, radioisotope assay, viscometric assay, coagulation assay, clotting time assay, protein synthesis assay, histological assay, culture assay, osmolarity assay, and/or other types of assays, centrifugation, separation, filtration, dilution, enriching, purification, precipitation, pulverization, incubation, pipetting, transport, cell lysis, or other sample preparation action or steps, or combinations thereof. Steps, tests, or assays that may be prepared or run by the device may include imaging, including microscopy, cytometry, and other techniques preparing or utilizing images. Steps, tests, or assays that may be prepared or run by the device may further include an assessment of histology, morphology, kinematics, dynamics, and/or state of a sample, which may include such assessment for cells.

A sample analysis device may be capable of performing all on-board steps (e.g., steps or actions performed by a single device) in a short amount of time. A sample analysis device may be capable of performing all on-board steps on a single sample in a short amount of time. For example, from sample collection from a subject to transmitting data and/or to analysis may take about 3 hours or less, 2 hours or less, 1 hour or less, 50 minutes or less, 45 minutes or less, 40 minutes or less, 30 minutes or less, 20 minutes or less, 15 minutes or less, 10 minutes or less, 5 minutes or less, 4 minutes or less, 3 minutes or less, 2 minutes or less, or 1 minute or less. The amount of time from accepting a sample within the device to transmitting data and/or to analysis from the device regarding such a sample may depend on the type or number of steps, tests, or assays performed on the sample. The amount of time from accepting a sample within the device to transmitting data and/or to analysis from the device regarding such a sample may take about 3 hours or less, 2 hours or less, 1 hour or less, 50 minutes or less, 45 minutes or less, 40 minutes or less, 30 minutes or less, 20 minutes or less, 15 minutes or less, 10 minutes or less, 5 minutes or less, 4 minutes or less, 3 minutes or less, 2 minutes or less, or 1 minute or less.

A sample analysis device may be configured to prepare a sample for disposal, or to dispose of a sample, such as a biological sample, following processing or assaying of a sample.

In embodiments, a sample analysis device may be configured to transmit data obtained from a sample. In embodiments, a sample analysis device may be configured to communicate over a network. A sample analysis device may include a communication module that may interface with the network. A sample analysis device may be connected to the network via a wired connection or wirelessly. The network may be a local area network (LAN) or a wide area network (WAN) such as the Internet. In some embodiments, the network may be a personal area network. The network may include the cloud. The sample analysis device may be connected to the network without requiring an intermediary device, or an intermediary device may be required to connect a sample analysis device to a network. A sample analysis device may communicate over a network with another device, which may be any type of networked device, including but not limited to a personal computer, server computer, or laptop computer; personal digital assistants (PDAs) such as a Windows CE device; phones such as cellular phones, smartphones (e.g., iPhone, Android, Blackberry, etc.), or location-aware portable phones (such as GPS); a roaming device, such as a network-connected roaming device; a wireless device such as a wireless email device or other device capable of communicating wireless with a computer network; or any other type of network device that may communicate possibly over a network and handle electronic transactions. Such communication may include providing data to a cloud computing infrastructure or any other type of data storage infrastructure which may be accessed by other devices.

A sample analysis device may provide data regarding a sample to, e.g., a health care professional, a health care professional location, such as a laboratory, or an affiliate thereof. One or more of a laboratory, health care professional, or subject may have a network device able to receive or access data provided by the sample analysis device. A sample analysis device may be configured to provide data regarding a sample to a database. A sample analysis device may be configured to provide data regarding a sample to an electronic medical records system, to a laboratory information system, to a laboratory automation system, or other system or software. A sample analysis device may provide data in the form of a report.

A laboratory, device, or other entity or software may perform analysis on data regarding a sample in real-time. A software system may perform chemical analysis and/or pathological analysis, or these could be distributed amongst combinations of lab, clinical, and specialty or expert personnel. Analysis may include qualitative and/or quantitative evaluation of a sample. Data analysis may include a subsequent qualitative and/or quantitative evaluation of a sample. Optionally, a report may be generated based on raw data, pre-processed data, or analyzed data. Such a report may be prepared so as to maintain confidentiality of the data obtained from the sample, the identity and other information regarding the subject from whom a sample was obtained, analysis of the data, and other confidential information. The report and/or the data may be transmitted to a health care professional. Data obtained by a sample analysis device, or analysis of such data, or reports, may be provided to a database, an electronic medical records system, to a laboratory information system (LIS), to a laboratory automation system (LAS), or other system or software.

A sample may be placed in a sample container, e.g., a vial, tube, or other container configured to hold a sample. A sample container may be configured to fit into, or be held by, a cartridge. For example, a cartridge may include a sample in a sample container, and may also include reagents for use in processing or testing a sample, disposables for use in processing or testing a sample, or other materials. A cartridge may be configured for placement on, or insertion into, a sample analysis device. In embodiments, one or more components of the cartridge may be brought into fluid communication with other components of the sample analysis device. For example, one or more reagents may be transferred to portions of the sample analysis device, and may be used in the analysis of a sample.

Samples collected in a sample collection room may be placed in a sample transport container. A sample transport container may be located in, or may located near to, a sample collection room. In embodiments, a sample collection room as disclosed herein may include a sample transport container. In embodiments, a system comprising a sample collection room as disclosed herein may include a sample transport container. In embodiments, samples may be placed in a sample transport container, and then soon or immediately transported to another location. In embodiments, samples may be stored in a sample transport container for a period of time after collection, and may be transported to another location for analysis, inspection, recordation, storage, or for other purposes. A sample transport container may be insulated, or may be refrigerated, or both.

A sample transport container may be configured to hold a single sample, e.g., a single sample within a single sample container; or may be configured to hold a plurality of samples, e.g., a plurality of samples each in a single sample container. In embodiments, such samples are fluid samples, such as blood or urine samples. In embodiments, such samples have small volumes, typically less than about 500 μL. A sample transport container may be configured to protect the samples, and sample containers, during transport. For example, a sample transport container may be configured to protect the samples, and sample containers, from physical shock, thermal changes, desiccation, or other damage which may occur during transport. A sample transport container may include padding; may include insulation; may include layers, including one or more inner containers, and one or more outer containers, configured to protect and isolate the samples held by the sample transport container. A sample transport container may include a thermal control unit, and may include material that provides active and/or passive cooling. For example in addition to, or in place of, insulating material, a sample transport container may include phase change material (PCM) material that maintains the temperature at a prior, or desired temperature. A sample transport container may include or use an active cooling system. Optionally, a sample transport container may use an active cooling system to keep and/or extend cooling time associated with a passive cooling component.

A sample transport container may include a temperature indicator, and may include a thermal change indicator. For example a temperature indicator or thermal change indicator may be disposed on a visible surface of the transport container. A temperature indicator may include a thermometer, or a display or read-out (e.g., of a thermocouple, thermistor, or other temperature sensor) configured to display the temperature within the sample transport container. A thermal change indicator may display a message, or alert, to indicate if or when the temperature within the sample transport container has exceeded a pre-defined limit. For example, thermochromic ink may be used as an indicator of thermal change, particularly if the thermal change resulted in temperatures outside a desired range.

It is to be understood that both the foregoing general description and the following detailed description are exemplary and explanatory only and are not restrictive of the invention, as claimed. It may be noted that, as used in the specification and the appended claims, the singular forms “a”, “an” and “the” include plural referents unless the context clearly dictates otherwise. Thus, for example, reference to “a material” may include mixtures of materials, reference to “a compound” may include multiple compounds, and the like. References cited herein are hereby incorporated by reference in their entirety, except to the extent that they conflict with teachings explicitly set forth in this specification.

In this specification and in the claims which follow, reference will be made to a number of terms which shall be defined to have the following meanings:

“Optional” or “optionally” means that the subsequently described circumstance may or may not occur, so that the description includes instances where the circumstance occurs and instances where it does not. For example, if a device optionally contains a feature for a sample collection unit, this means that the sample collection unit may or may not be present, and, thus, the description includes both structures wherein a device possesses the sample collection unit and structures wherein sample collection unit is not present.

As used herein, the terms “substantial” means more than a minimal or insignificant amount; and “substantially” means more than a minimally or insignificantly. Thus, for example, the phrase “substantially different”, as used herein, denotes a sufficiently high degree of difference between two numeric values such that one of skill in the art would consider the difference between the two values to be of statistical significance within the context of the characteristic measured by said values. Thus, the difference between two values that are substantially different from each other is typically greater than about 10%, and may be greater than about 20%, preferably greater than about 30%, preferably greater than about 40%, preferably greater than about 50% as a function of the reference value or comparator value.

As used herein, the term “point of service location” (POS) may include locations where a subject may receive a service (e.g. testing, monitoring, treatment, diagnosis, guidance, sample collection, ID verification, medical services, non-medical services, etc.), and may include, without limitation, a subject's home, a subject's business, the location of a healthcare provider (e.g., doctor), hospitals, emergency rooms, operating rooms, clinics, health care professionals' offices, laboratories, retailers [e.g. pharmacies (e.g., retail pharmacy, clinical pharmacy, hospital pharmacy), drugstores, supermarkets, grocers, etc.], transportation vehicles (e.g. car, boat, truck, bus, airplane, motorcycle, ambulance, mobile unit, fire engine/truck, emergency vehicle, law enforcement vehicle, police car, or other vehicle configured to transport a subject from one point to another, etc.), traveling medical care units, mobile units, schools, day-care centers, security screening locations, combat locations, health assisted living residences, government offices, office buildings, tents, bodily fluid sample acquisition sites (e.g. blood collection centers), sites at or near an entrance to a location that a subject may wish to access, sites on or near a device that a subject may wish to access (e.g., the location of a computer if the subject wishes to access the computer), a location where a sample processing device receives a sample, or any other point of service location described elsewhere herein.

As used herein, the terms “retail store”, “retail location”, “retailer”, and the like refer to places of business in which retail transactions typically take place; a retail store is primarily a location for business, and not for the practice of medicine or ancillary clinical procedures and activities. Thus, a retail store is not primarily a location at which clinical procedures, such as collection of samples for diagnosis or other clinical use, are performed. However, although not a primary purpose or activity, collection of clinical samples are one of the activities which may take place at a retail store. A retail store may be, without limitation, a pharmacy, including, e.g., a retail pharmacy, a clinical pharmacy, a hospital pharmacy, and a dispensary; a drugstore; a supermarket or grocery store; or other retail store.

As used herein, a “sample”, or “biological sample”, or “clinical sample” refers to a sample of fluid, tissue, secretion, or excretion obtained from a subject. A clinical sample may be a sample of blood, serum, plasma, saliva, sputum, urine, gastric fluid, digestive fluid, tears, sweat, stool, semen, vaginal fluid, interstitial fluid, fluid derived from tumorous tissue, ocular fluids, mucus, earwax, oil, glandular secretions, spinal fluid, skin, cerebrospinal fluid from within the skull, tissue, fluid or material from a nasal swab, a throat swab, a cheek swab, or nasopharyngeal wash, biopsy fluid or material, placental fluid, amniotic fluid, cord blood, lymphatic fluids, cavity fluids, pus, microbiota obtained from a subject, meconium, breast milk, or other secretion or excretion. A sample may be a breath sample, a hair sample, a fingernail sample, or other sample.

Biological samples may include nasopharyngeal wash, or other fluid obtained by washing a body cavity or surface of a subject, or by washing a swab following application of the swab to a body cavity or surface of a subject. Nasal swabs, throat swabs, cheek swabs, vaginal swabs, cervical swabs, stool samples, hair, finger nail, ear wax, breath, and other solid, semi-solid, or gaseous samples may be processed in an extraction buffer, e.g., for a fixed or variable amount of time, prior to their analysis. The extraction buffer or an aliquot thereof may then be processed similarly to other fluid samples if desired. Examples of tissue samples of the subject may include but are not limited to, connective tissue, muscle tissue, nervous tissue, epithelial tissue, cartilage, cancerous sample, or bone. The sample may be obtained from a human or animal. The sample may be obtained from a vertebrate, e.g., a bird, fish, or mammal, such as a rat, a mouse, a pig, an ape, another primate (including humans), a farm animal, a sport animal, or a pet. The sample may be obtained from a living or dead subject. The sample may be obtained fresh from a subject or may have undergone some form of pre-processing, storage, or transport.

Thus, as used herein, a “sample” may be, but is not limited to, a fluid sample, such as a blood or urine sample, or a portion of a blood or urine sample. A sample may be of any suitable size or volume, and is preferably of small size or volume. In some embodiments of the assays and methods disclosed herein, measurements may be made using a small volume blood sample, or no more than a small volume portion of a blood sample, where a small volume comprises no more than about 5 mL; or comprises no more than about 3 mL; or comprises no more than about 2 mL; or comprises no more than about 1 mL; or comprises no more than about 500 μL; or comprises no more than about 250 μL; or comprises no more than about 100 μL; or comprises no more than about 75 μL; or comprises no more than about 50 μL; or comprises no more than about 35 μL; or comprises no more than about 25 μL; or comprises no more than about 20 μL; or comprises no more than about 15 μL; or comprises no more than about 10 μL; or comprises no more than about 8 μL; or comprises no more than about 6 μL; or comprises no more than about 5 μL; or comprises no more than about 4 μL; or comprises no more than about 3 μL; or comprises no more than about 2 μL; or comprises no more than about 1 μL; or comprises no more than about 0.8 μL; or comprises no more than about 0.5 μL; or comprises no more than about 0.3 μL; or comprises no more than about 0.2 μL; or comprises no more than about 0.1 μL; or comprises no more than about 0.05 μL; or comprises no more than about 0.01 μL.

As used herein, a “small volume” refers to a volume of less than about 1 mL, or less than about 500 μL, or less than about 250 μL, or less than 150 μL, or less than about 100 μL, or less than about 50 μL, or less than about 25 μL, or less. In particular embodiments, a small volume, such as a “finger-stick” volume, may comprise less than about 250 μL, and typically comprises less than 150 μL, or less than about 100 μL, or less than about 50 μL, or less than about 25 μL, or less.

As used herein, a “short period of time” refers to a period of time of about 5 hours or less, or about 4 hours or less, or about 3 hours or less, or about 2 hours or less, or about 1 hour or less, or about 50 minutes or less, or about 40 minutes or less, or about 30 minutes or less, or about 20 minutes or less, or about 10 minutes or less, or about 5 minutes or less. A short period of time may be determined with respect to an initial time; the initial time may be the time at which a sample analysis began; the initial time may be the time at which a sample is inserted into a device for the analysis of the sample; the initial time may be the time at which a sample was obtained from a subject.

It should be understood that embodiments in this disclosure may be adapted to have one or more of the features described below.

Systems, methods, and devices for obtaining samples are provided. Such samples are provided for analysis in order to detect a diseases or condition of a subject. Such samples may be used, for example, to detect the presence of markers indicative of one or more of a plurality of infectious agents in a single clinical sample, or in a plurality of aliquots of a single clinical sample. The systems, methods, and devices disclosed herein may be point-of-service systems, methods, and devices, configured for use at a point-of-service location, where a point-of-service location may be a location at which a sample is obtained from a subject. In the embodiments disclosed herein, a sample is obtained from a subject at a retail location. In particular, a sample is obtained from a subject at a designated sample collection location within a retail location.

In embodiments, the sample is a small-volume sample. In embodiments, the sample is used to test for a plurality of markers, or a plurality of diseases; such testing may include nucleic acid testing (e.g., testing for DNA or RNA sequences present in the sample), amino acid testing (e.g., using antibodies or binding proteins), general chemistry testing, cytometric testing (e.g., producing and analyzing images of cells in a sample, or using other optical and microscopic techniques), and other testing. In embodiments, the sample is collected at the retail location, and is analyzed at another location. In embodiments, the analysis of the small-volume clinical sample is completed in a short period of time. In embodiments, the sample is collected at the retail location, and is analyzed in a device at the retail location. In embodiments, the analysis of the small-volume clinical sample is completed in a short period of time.

Methods for obtaining a sample from a subject include providing a sample collection room within a retail store; obtaining a sample from the subject; and analyzing the sample. Samples may be collected in a sample collection room adjacent a waiting room; a bathroom may be provided with a pass-through to the sample collection room. A sample may be sent to a remote location for analysis. A sample may be analyzed at, or adjacent to, the sample collection location. A sample collection room may house a sample analysis device or sample analysis system, and thus also serve as a sample analysis room. A sample may be a small sample, e.g., a small blood sample may be taken from a single finger-stick, or two or a few finger-sticks. A sample may be analyzed in a short period of time, e.g., in less than five hours, or less than four hours.

Methods for obtaining a sample from a subject include providing a sample collection room within a retail store, and obtaining a sample from the subject.

Methods for analyzing a sample obtained from a subject include providing a sample collection room within a retail store; obtaining a sample from the subject; and analyzing the sample. In embodiments of methods for analyzing a sample obtained from a subject the sample is analyzed at the retail location. A sample collection room may be adjacent a waiting room; a bathroom may be provided; a pass-through from bathroom to sample collection room may be provided.

A warming table may be provided in a sample collection room which has an integrated passthrough window specially designed to transfer urine into the blood collection area and into sample analysis devices or systems.

In embodiments of methods for obtaining a blood sample from a subject, blood may be obtained from a small skin puncture. Such a small skin puncture may be, e.g., a small skin puncture on a finger, such as on a finger tip; a small skin puncture on a toe, such as on or near a tip of a toe; a small skin puncture on a foot, such as on or near the heel; or other small skin puncture. A small skin puncture is on a finger may be termed a “finger-stick”; the term finger-stick may also be used to describe the volume of a sample, i.e., a finger-stick volume is one, two, or a few drops (e.g., about 50 μL to about 300 μL, or about 75 μL to about 250 μL, or about 80 μL to about 150 μL).

In embodiments, a sample collection room includes a location for a practitioner (e.g., a sample collection technician) to sit and obtain a bodily fluid or other sample from the patient. In embodiments, a sample collection room includes a stool; a stool may have wheels; may have a back; and may be adjustable in height.

A work surface such as a table is provided so that a patient has a surface on which they can position the target site on the subject to allow for sampling. A work surface such as a table in a sample collection room as disclosed herein may be movable, and may have wheels, effective that it may be positioned near to a subject for support of a finger, hand, arm, leg, foot, heel, toe, or other target body site for sample collection from a subject. In embodiments, a work surface such as a table may be attachable to a chair on which a subject reposes during sample collection. In embodiments, the height, angle, or other orientation of a work surface such as a table may be adjusted for sample collection; such adjustment may aid in access to a target body site; may improve the subject's comfort prior to, during, or after sample collection; may improve blood flow during sample collection; and may have other advantages. In embodiments, a work surface may include or be adjacent to storage (e.g., may be part of a movable table having a drawer; or may be positioned on top of a drawer, or shelf, of a movable cabinet). Such storage may be used to hold supplies and implements used in sample collection (e.g., lancets, sample containers, tubes, gauze, or other items). In embodiments, the work surface storage not only contains sample collection implements and supplies, but is effective to conceal them from the subject at most times prior to and during sample collection. In embodiments, the work surface comprises a surface that is easily cleaned, and that may be sterilized. A work surface may be or include a hard surface (e.g., plastic, glass, hard rubber, acrylic, polymer, or other material) or may be or include a soft or padded surface (e.g., cloth, rubberized cloth, soft rubber, or other material).

The work surface may have one or more thermal controlled sites wherein the temperature of the target site on the patient may be brought to a desired temperature. The temperature may be adjustable. By way of example and not limitation, embodiments may heat a finger or other target site to improve blood flow and thus blood yield from a finger-stick. For example, the table may have thermal control areas to increase blood flow to the target area and thus increase the speed with which sufficient blood or other bodily fluid can be drawn from the subject. The heating is used to bring the target tissue to between about 40° C. to about 50° C. In embodiments, the heating brings target tissue to within a temperature range of between about 40° C. to about 44° C. In embodiments, the heating brings target tissue to within a temperature range of between about 41° C. to about 43° C. In embodiments, the heating brings target tissue to a temperature of about 42° C. In embodiments, the heating brings target tissue to within a temperature range of between about 44° C. to about 47° C. In one embodiment, the temperature is sufficient to increase blood flow to yield 120 uL of sample. In one embodiment, the temperature is sufficient to increase blood flow to yield 130 uL of sample. In one embodiment, the temperature is sufficient to increase blood flow to yield 140 uL of sample. In one embodiment, the temperature is sufficient to increase blood flow to yield 150 uL of sample. Optionally, the thermal controlled site is a shaped surface is contoured to match that of the target site on a patient.

In embodiments, the thermal controlled site comprises a hard surface that is easily cleaned, and that may be sterilized. Such a surface may be or include, for example, plastic, glass, hard rubber, acrylic, polymer, and other materials. In embodiments, the thermal controlled site comprises a soft or padded surface that is easily cleaned, and that may be sterilized. Such a surface may be or include, for example, cloth, rubberized cloth, soft rubber, and other materials.

In embodiments, such a temperature-controlled work surface is provided as part of a table, which may be termed a warming table. Accordingly, a sample collection room may include a warming table. A warming table may be configured to warm a subject's hand, foot, or other body part. The warming table may be tiltable; and its height may be adjustable. A warming table may be configured for use with the left hand, or right hand, of a subject. A warming table may be configured for use with the left foot, or right foot, of a subject. For example, a warming table may be configured for placement near a chair (such as a reclining chair) on either side of a subject seated in the chair.

It will be understood that a warming table may be any fixture or piece of furniture which provides a temperature-controlled surface on which a subject may place a part of their body (e.g., hand, arm, foot, or other part) for obtaining a sample. Thus, a warming table may provide such a temperature-controlled surface configured to receive a part of a subject's body; may provide such a temperature-controlled surface and also provide storage, e.g., for supplies; may provide such a temperature-controlled surface and also provide guides for placement of a hand, arm, foot, or other part of a subject's body; may provide such a temperature-controlled surface and also provide lighting, e.g., for illuminating the subject or a part of a subject's body; a waste receptacle; and other features and elements, and combinations thereof. Supply storage may be provided by drawers, shelves, bins, slots, racks, or other features. A waste receptacle may be configured to receive any waste, may be configured to receive biohazard waste (e.g., waste containing bodily fluids or tissues); or both.

In embodiments, a warming table is adjustable, effective to adjust the temperature of a surface of the warming table (e.g., a surface in contact with a body part of a subject from whom a sample is, or is to be, obtained). In embodiments, a warming table is adjustable, effective to adjust the height of the warming table, including effective to adjust the height of a warming surface. In embodiments, a warming table is adjustable, effective to adjust the orientation, or the tilt, of the warming table. In embodiments, a warming table is adjustable, effective to adjust the distance of the warming table (or a surface thereof) from a subject. In embodiments, a warming table is adjustable, effective to adjust the presence of, position of, and orientation of, a rest, such as a hand or arm rest, on or near the warming table.

In embodiments, a warming table is configured to place the hand or other body part from which a blood sample is to be collected at an optimal height and orientation for facilitating the flow of gravity in the finger as blood droplets form. A warming table may include a rest, such as an arm rest, or hand rest, or other rest which provides an additional surface for contacting a subject; such a surface may make positioning of a hand, or arm, or foot, or leg, other body part more comfortable for the subject, more convenient for the sample collection technician, or both. Such a rest may be configured for placement in more than one position or orientation. Such a rest may be retractable or otherwise configured to be placed or stored out of the way of a subject or sample collection technician when not needed. A warming table may be designed to accommodate the seating or placement of a sample collection technician near to a subject. A warming table, or surface thereof, may be adjustable, or may include an adjustable surface, which may be placed or oriented for the comfort and convenience of a subject, a sample collection technician, or both. For example, a warming table may include a sliding surface which may be extended or retracted as needed to accommodate a subject or a sample collection technician; may include a tiltable surface which may be oriented as needed to accommodate a subject or a sample collection technician; may include a surface which may be raised or lowered as needed to accommodate a subject or a sample collection technician; or be otherwise adjustable.

Accordingly, one or more of the lateral position, the height, and the orientation (e.g., tilt or camber) of a warming table, or a surface thereof, may be adjustable. For example, the height of an upper surface, or portion thereof, of a warming table may be adjustable, e.g., to accommodate the comfort or convenience of the subject, of the sample collection technician, or both. Similarly, the orientation of an upper surface, or portion thereof, of a warming table may be adjustable, e.g., to accommodate the comfort or convenience of the subject, of the sample collection technician, or both. In embodiments, the upper surface of a warming table, or a portion thereof, may be configured for easy cleaning and for maintaining sterility during sample collection. The upper surface of a warming table may be padded, or include a padded portion. The upper surface of a warming table may be water-proof, or water-resistant, or include a water-proof, or water-resistant portion.

In embodiments, the height (as measured from the floor) of the upper surface of a warming table configured to receive a part of a subject's body (e.g., during sample collection) may be between about 10 inches and about 40 inches; or between about 15 inches and 30 inches; or between about 16 inches and 28 inches; or between about 20 inches and about 24 inches in height. In embodiments, the length of a warming table may be between about 20 inches and about 60 inches; or between about 24 inches and 54 inches; or between about 20 inches and 50 inches; or between about 35 inches and about 45 inches in length. In embodiments, the width of a warming table may be between about 8 inches and about 30 inches; or between about 10 inches and 24 inches; or between about 12 inches and 18 inches; or between about 11 inches and about 15 inches in width. In embodiments, a warming table may have a length of about 38 to 42 inches, a width of 12 to 15 inches, and a height of about 19 to about 23 inches.

In embodiments, the orientation of the upper surface of a warming table configured to receive a part of a subject's body (e.g., during sample collection) may be substantially parallel to the floor of the sample collection room.

A subject may sit or lie next to a warming table during sample collection. When seated or lying next to a warming table, the subject presents a subject orientation axis extending from the subject's head towards the subject's feet. The orientation of the upper surface of a warming table configured to receive a part of a subject's body (e.g., during sample collection) may be adjustable; in embodiments, such orientation may be adjustable along an axis substantially parallel to the subject orientation axis; or may be adjustable along an axis substantially perpendicular to the subject orientation axis. In embodiments, the orientation of the upper surface of a warming table may be adjustable along two axes (e.g., along an axis substantially parallel to, and along an axis substantially perpendicular to, the subject orientation axis). It will be understood that the orientation of an upper surface of a warming table may be adjustable along other axes, or in other ways, as well as or instead of along these described axes. In embodiments, the orientation of the upper surface of a warming table may be adjustable between angles between about 0° to about 90°, or between about 0° to about 45°, or between about 0° to about 30°, or other ranges (where 0° indicates level, i.e., substantially parallel to the floor).

It will be understood that adjustment of the height, or orientation, or both, of an upper surface of a warming table may comprise adjustment of a portion of the warming table other than the upper surface; for example, adjustment of the height or orientation of an upper surface of a warming table may be effected by adjusting a leg, or legs, or the warming table, or by adjusting the surface (e.g., the floor or a portion of the floor, or other surface on the floor) on which the warming table is placed; by adjusting an upper portion of the table; by adjusting a lower portion of the table; by adjusting an intermediate portion of the table; or combinations thereof.

A warming table may be used to warm the fingers of a subject prior to puncturing a finger to obtain a blood sample. The thermal regulation of the warming table may be used in conjunction with a finger warmer (a portable device for placement on or around a finger, fingers, or hand of a subject. The thermal regulation of the warming table may be used in place of a finger warmer. In embodiments, a warming table may include a built-in waste receptacle. In embodiments, such a built-in waste receptacle may be, or may include, a biohazard bin.

A sample collection room may include a chair. In embodiments, the chair may be a reclining chair. The chair may be configured for ease of placement of an arm or hand of a subject effective to place the hand of the subject on a warming table, e.g., in proper placement on a warming table for effective warming of the hand prior to obtaining a blood sample. In embodiments, a chair in a sample collection room as disclosed herein may have and arm, or arms, suitable for comfortably supporting an arm of a subject; may be able to recline to allow a subject in the chair to lay back for comfort, ease, and safety; and may be accessible by a technician from both sides (e.g., configured so that a left arm or hand, and a right arm or hand of a subject are equally accessible to a technician for obtaining a sample). Where a foot of the subject is to be used for obtaining a sample, the chair may be configured for ease of placement of a leg or foot of a subject effective to place the foot of the subject on a warming table, e.g., in proper placement on a warming table for effective warming of the foot prior to obtaining a blood sample. Sample collection from a leg or foot may be preferred, for example, for pediatric patients, or amputees, or patients with impaired blood flow to a hand, or other subjects. A booster, pillow, insert, or other item or feature may be used to position a pediatric or other patient in proper position for obtaining the sample. In embodiments, a chair in a sample collection room as disclosed herein may be placed in a corner of a sample collection room. In embodiments, a chair in a sample collection room as disclosed herein may be colored ivory, white, or off-white, or other neutral color; may have arms or arm-rests made at least partially of wood (e.g., birch wood, pine, or other wood); and may have a leg-rest made at least partially of wood (e.g., birch wood, pine, or other wood).

Such a chair may be placed adjacent a warming table, effective that a subject may comfortably place their hand, or other body part to be used for sample collection, on the warming table. In embodiments, the warming table is movable. In embodiments, the chair and the warming table are configured for use together. In embodiments, the chair is connected to the warming table. In embodiments, the chair and the warming table form a single unit. In embodiments, the chair and the warming table form a single unit after connection (e.g., latching, locking, or otherwise securing) of the chair and warming table together. In embodiments, the chair and the warming table are modified to become a single unit (e.g., by screws, nails, clamps, or other permanent or semi-permanent fastening). In embodiments, the chair and the warming table are built as a single unit.

A sample collection room may include a refrigerator. In embodiments, a sample collection room may include two refrigerators, or may include a plurality of refrigerators. A refrigerator may be placed in a ventilated cabinet. In embodiments, a refrigerator is placed at a level that is convenient for ready access by a technician (e.g., for retrieval of materials or supplies, or for placement of a sample following collection, or for other uses). In embodiments, counter height is a convenient height for the placement of a refrigerator. In embodiments, a refrigerator may be placed to the right of a work surface (e.g., a table, or a warming table, or other surface on which a subject may rest a target body site for sample collection is placed to the technician's right when the technician is seated in place for sample collection). In embodiments of sample collection rooms including two or more refrigerators, one or more refrigerators may be designated for, and may be used for, storage of samples following sample collection. In embodiments of sample collection rooms including one or more refrigerators that are designated for, or used for, storage of samples, such one or more refrigerators may be designated “BioHazard” refrigerators. In embodiments of sample collection rooms including two or more refrigerators, one or more refrigerators may be designated for, and may be used for, storage of food, drink, reagents, containers, and consumables for use in sample collection or sample analysis. In embodiments of sample collection rooms including one or more refrigerators that are designated for, or used for, storage of food, drink, reagents, containers, and consumables, such one or more refrigerators may be designated “Clean” refrigerators. In embodiments, food or drink stored in a refrigerator in a sample collection room may be offered to a subject, e.g., before collection of a sample from the subject, or after collection of a sample from the subject, or both. In embodiments, food or drink stored in a refrigerator in a sample collection room may be useful for sample collection or analysis; e.g., where a glucose tolerance test is to be performed, a subject may be given a drink or food containing glucose prior to sample collection.

In embodiments, a sample collection room may include a centrifuge. In embodiments, a centrifuge included in a sample collection room may be configured to receive and operate upon (e.g., spin effective to apply centrifugal force to) a small sample collected in the sample collection room. In embodiments, a centrifuge included in a sample collection room may be configured to receive a sample container holding a small sample collected in the sample collection room. In embodiments, a centrifuge included in a sample collection room may be configured to receive a plurality of sample containers, each holding a small sample collected in the sample collection room. In embodiments, a centrifuge included in a sample collection room may be configured to receive one, or a plurality of, calibration containers, each holding a small calibration reagent. In embodiments, a centrifuge included in a sample collection room may be configured for placement on, and for use on, a table, a counter-top, or other work surface.

In embodiments, a sample collection room may include a computer, and may include one or more of a computer keyboard, mouse, and monitor (which may be, e.g., operably connected to a computer). In embodiments, a sample collection room may include a scanner, reader, or other device i) capable of reading an optical code such as a bar-code, a QR-code, image displayed on a cell-phone, or other optical label or optical code; ii) capable of reading a magnetic code such as a magnetic strip, or other label or code; iii) capable of reading a radiofrequency identification (RFID) label or code; or iv) other label or code related to a sample, a container, a reagent, a kit, an individual patient (subject), or other labeled material. In embodiments, a sample collection room may include a bar-code printer, or label printer, or other device suitable for preparing a label, tag, sign, or other identifier, e.g., for affixing to a sample collected in the sample collection room.

Calming features may be selected from flowing water, flowers, plants, scents, sounds, colors, images, and other features and elements which may reduce any anxiety felt by a subject prior to, during, or after obtaining a sample. A flower may be a cut flower, a flower growing as part of a plant, an artificial flower, or an image of a flower.

A sample collection room may include a calming feature. For example, a calming feature may include flowing water (e.g., a wall fixture with flowing water, such as water flowing down from an upper location along a face to a lower collecting trough or frame; a fountain; a rock or rocks over which water flows; an artificial stream; and other flowing water features). A calming feature, including a water feature, may also include a plant, or plants, including ferns, bamboo, moss, flowering plants (e.g., orchids, roses, and other plants), bonsai trees and plants, and other plants. A water feature may produce calming sounds.

A calming feature may include calming sounds in addition to, or exclusive of, a water feature; for example, speakers may provide recorded or synthesized music or sounds. Such music will typically be calming music designed to calm, to reduce anxiety, and to produce or enhance tranquility in a subject. Such sounds may include white noise sounds, beach sounds, forest sounds, bird sounds, traffic sounds, and other sounds, and combinations thereof.

A calming feature may include calming scents, which may be provided by candles present in the sample collection room, or may be provided by wicks, pads, tapes, oils, aerosols, or other means present in the sample collection room or connected to vents and air-channels connected to the sample collection room. Such calming scents may include perfumes, aromas, and other scents and scented materials, whether natural, artificial, or combinations thereof. Calming scents include, e.g., vanilla, rose scent, lavender, coconut, marjoram, chamomile, lilac, citrus scents, and others. Such scents may be provided by flowers (e.g., rose, jasmine, geraniums, and others) or plants (e.g., sage, mint, rosemary, and others) present in the sample collection room or placed in or near sources of airflow into the sample collection room.

In embodiments, the walls, furniture, fixtures, and other elements of a sample collection room may be white or off-white. In embodiments, the walls, furniture, fixtures, and other elements of a sample collection room may be colored in calming colors, e.g. light pastel colors, including greens, blues, yellows, and other light colors. In embodiments, black, red, purple, and other colors may be avoided. In embodiments, the walls and fixtures of a sample collection room may be white or off-white, and may include accents or designs in green or other calming colors. In embodiments, signs, posters, and other wall hangings (including video monitors and flat-screen monitors) may include still or moving images of water (e.g., streams, bays, or ocean images), and may include images of coral, kelp, or other water-living plants and animals (e.g., fish, including jelly fish) or other aquatic images. In embodiments, signs, posters, and other wall hangings (including video monitors and flat-screen monitors) may include still or moving images of plants, including garden, forest, and other pastoral images. In embodiments, a wall-mounted video monitor may provide continuous calming, aquatic-themed video images; such images may be accompanied by calming music or calming sounds related to, or complementary to, the video images.

Lighting in a sample collection room as disclosed herein is typically dimmer than sunlight, and may be soft white lighting; may be or include recessed lighting; may be or include indirect lighting; or may otherwise be configured to reduce glare and to conform with, and to enhance the calming effects of room design and features. In embodiments, soft white lighting in a sample collection room may be soft, white lighting of about 2000K to about 4000K; in embodiments, lighting in a sample collection room is soft, white lighting of about 3000K. A sample collection room may have adjustable lighting, so that the light intensity may be adjusted from dim to bright, and other levels in between, effective to provide a calming or reassuring effect on a subject.

In embodiments, flooring in a sample collection room as disclosed herein is typically configured to conform with, and to enhance the calming effects of room design and features. In embodiments, flooring in a sample collection room as disclosed herein may include light bamboo flooring.

A sample collection room as disclosed herein may include storage cabinets, or storage modules, or shelves, or tables (in addition to a table for supporting a target body site during sample collection), or other furniture suitable for storing supplies. Supplies which may be stored in a sample collection room as disclosed herein include supplies typically used in the course of sample collection (containers, lancets, tubes, pads, gauze, and other items and materials); informational literature and consent forms; replenishment supplies (e.g., pens, paper, ink, etc.); water bottles; and other supplies. Storage for technician personal items may also be provided, and may be accommodated with, or by, the storage cabinets, or storage modules, or shelves, or other furniture in a sample collection room as disclosed herein. In addition, a table, or shelf, or cabinet may be used to store, and may be used to display, brochures, informational literature, reading material, or other material for a subject.

A sample collection room may include a kiosk or work station configured to provide a sample collection technician with the supplies and equipment needed for sample collection in a location at, adjacent, or near to the collection location. For example, such a kiosk or work station may include, or be placed near to, a table configured for placing a limb of a subject in position for obtaining a sample. In embodiments, such a table may be a warming table.

A kiosk or work station (e.g., as disclosed in U.S. Patent Application 61/852,484 and in U.S. patent application Ser. No. 14/214,854, filed Mar. 15, 2014) may include a user accommodation section adapted to locate a user in at least one position that enables an interaction between the station and the user; a user interface adapted to permit a user to input data relevant to the user; wherein the at least one position enables the user to position a portion of their body onto a work surface for obtaining patient service. Such a workstation may include a secured, temperature controlled sample storage location that is configured to be accessible through a first opening; and a lockable cover for securing the user interface; in embodiments, the lockable cover may not secure the sample storage location. In embodiments, a secured, temperature controlled sample storage location may be configured to be accessible through a first opening and a second opening, wherein a key, code, or structure to open one opening does not open the other opening.

A sample may be sent to a remote location for analysis. A sample may be analyzed at, or adjacent to, the sample collection location. A sample collection room may house a sample analysis device or sample analysis system, and thus also serve as a sample analysis room. A sample may be a small sample, e.g., a small blood sample may be taken from a single finger-stick, or two or a few finger-sticks. A sample may be analyzed in a short period of time, e.g., in less than five hours, or less than four hours.

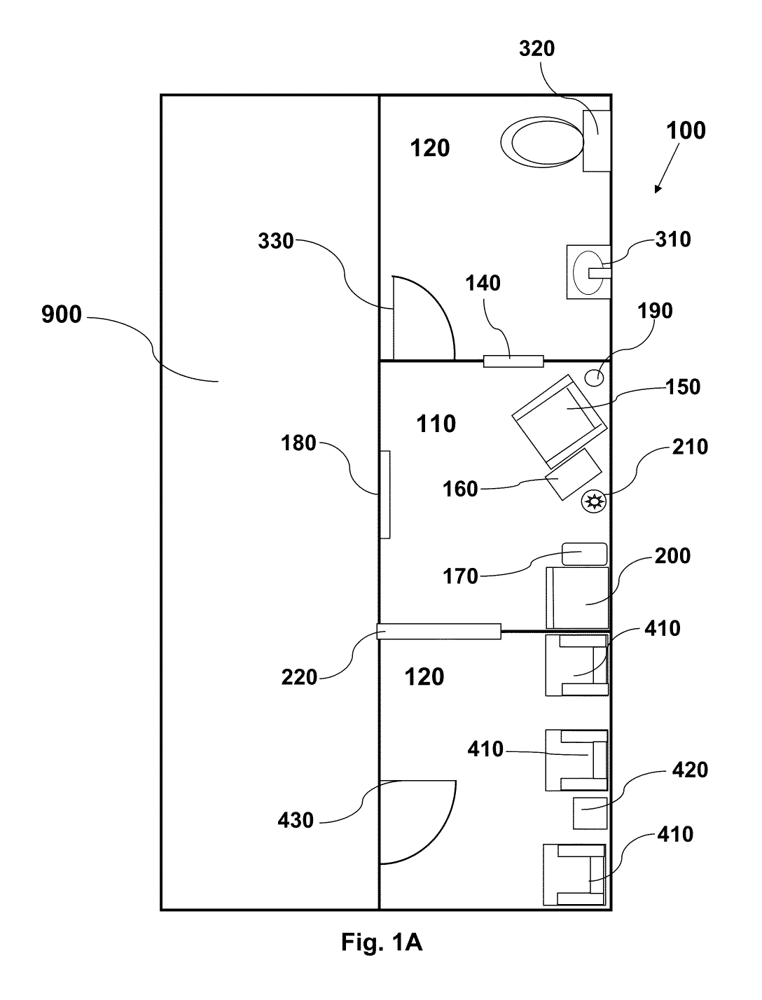

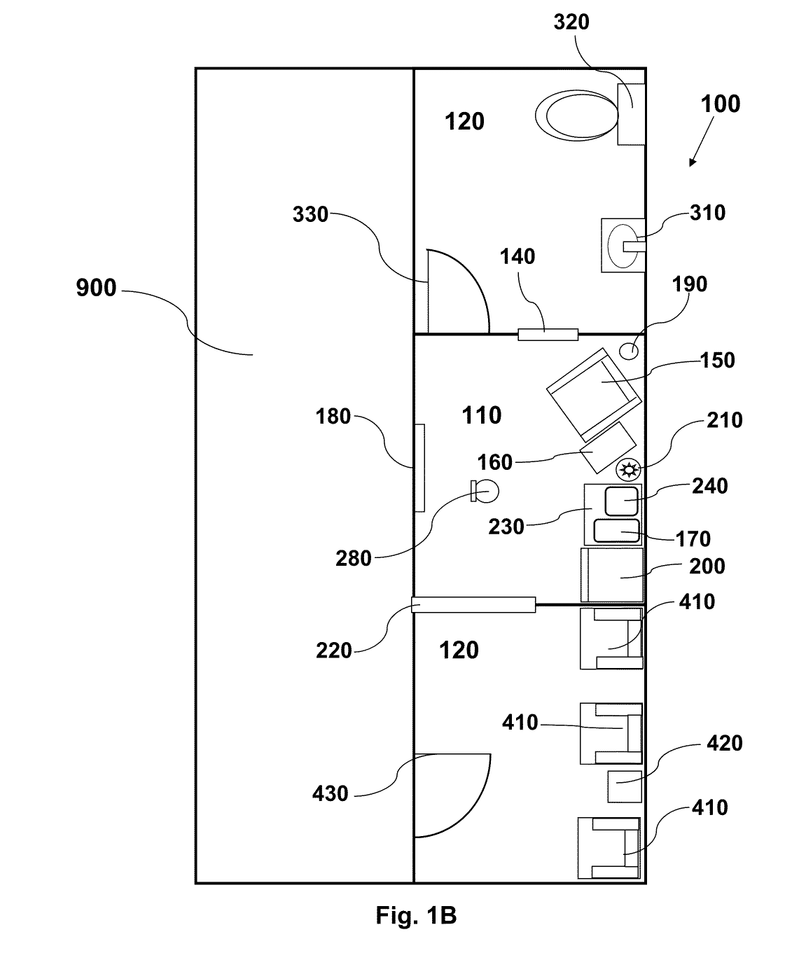

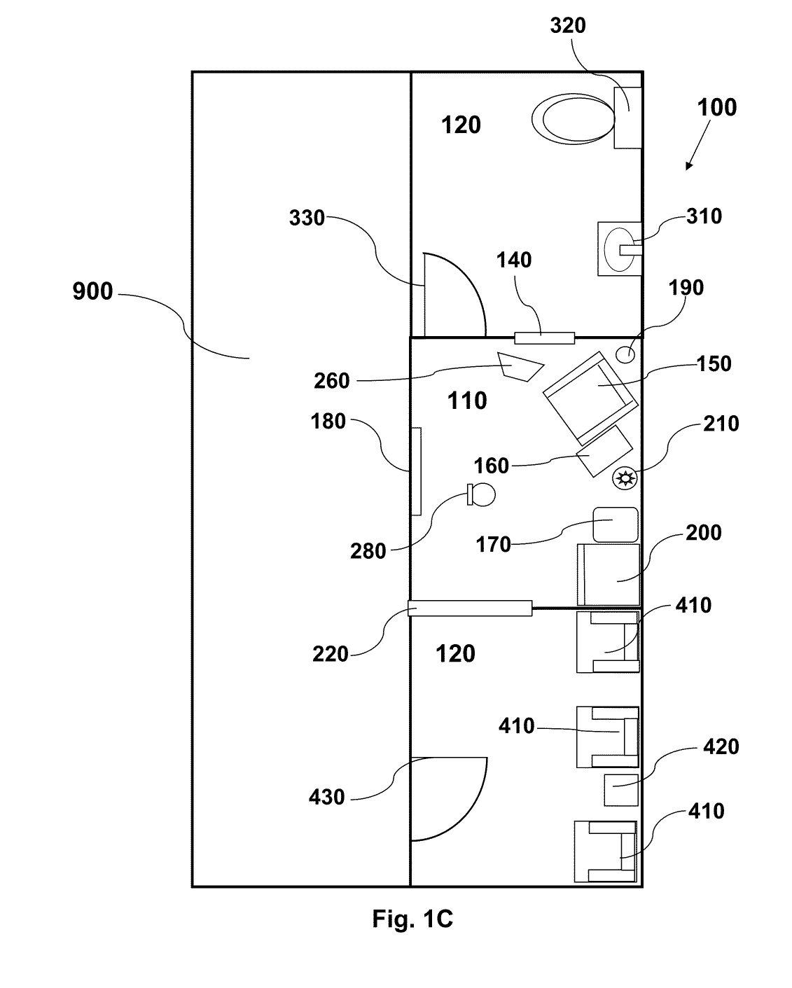

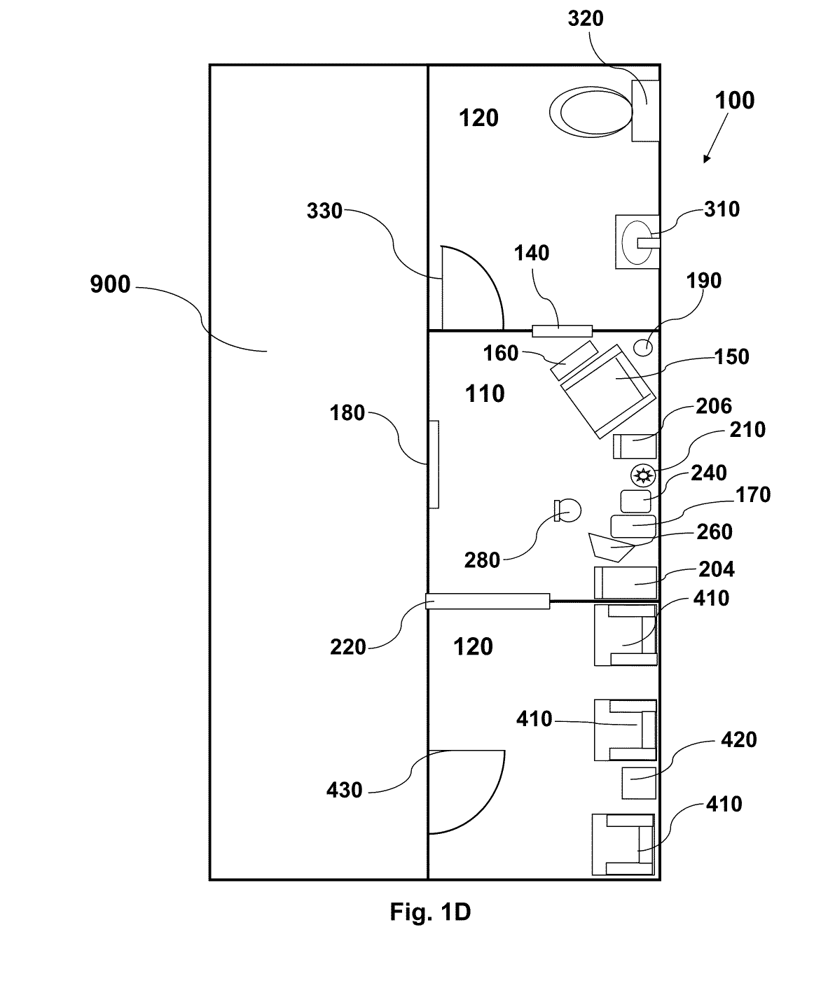

FIG. 1A shows an exemplary system comprising a sample collection center in a retail location, including a waiting room, a sample collection room, a bathroom, a pass-through connecting the bathroom with the sample collection room, and including an (optional) sample analysis device located in the sample collection room. As shown in FIG. 1A, a system may be a sample collection center 100 in a retail store (a portion of the store is indicated by 900). The sample collection center 100 includes a sample collection room 110, a waiting room 120, and a bathroom 130; sample collection room 110 and the bathroom 130 are connected by a pass-through 140, which may be used, e.g., to pass a urine, stool, or other sample from the bathroom 130 to the sample collection room 110. The sample collection room 110 includes a reclining chair 150; a warming table 160; an (optional) sample analysis device 170 for analyzing the sample at the sample collection center; a wall-hanging with flowing water 180; an adjustable lamp 190; a refrigerator 200; and a plant 210. In embodiments, a sample analysis device 170 may be placed on the floor, or on a table, or on a shelf, or in other convenient location. In embodiments, a refrigerator 200 may be used to store materials which should not be near to, or contaminate, or be contaminated by, samples collected in a sample collection room 110; such a refrigerator may be termed a “clean” refrigerator. In embodiments, a refrigerator 200 may be used to store samples collected in a sample collection room 110; since it contains material derived from humans or other living subjects, such a refrigerator may be termed a “biohazard” refrigerator (see FIG. 1D below which illustrates a clean refrigerator 204 and a biohazard refrigerator 206). In embodiments, a sample collection room 110 may include more than one refrigerator 200. In embodiments, a sample analysis device 170 may be placed on the floor, or on a table, or on a shelf, or in other convenient location. A movable stool 280 is provided for use by a technician, e.g., during sample collection, during sample analysis, or during performance of any other task. A technician may choose to seat themselves on stool 280, or may choose to stand during performance of sample collection, analysis, or other tasks. It will be understood that air flowing into the sample collection room 110 may be scented, and that soothing music or sounds may be provided in the sample collection room 110. Access to the sample collection room 110 from the waiting room 120 may be by door 220 (shown as a sliding door). Access to the waiting room 120 from retail store 900 may be via door 430. A subject may enter the bathroom 130 from the sample collection room 110 via door 330. Bathroom 130 includes a sink 310 and a toilet 320. Chairs 410 are placed in the waiting room 120 for the convenience of subjects waiting to have samples taken; a table 420 may provide space for informative literature or for light reading material for waiting subjects.

FIG. 1B shows a plan view of a sample collection room within a retail location, having a waiting room 120, a sample collection room 110, a centrifuge 240, a refrigerator 200, and a bathroom 130. Other elements shown are labeled as in FIG. 1A. Centrifuge 240 and sample analysis device 170 may be placed on a table 230 for convenience of access by a technician, who may be seated on a movable stool 280. In embodiments, a centrifuge 240 may be placed on a table 230 as shown, or may be placed on the floor, or on a shelf, or in other convenient location. In embodiments, a sample analysis device 170 may be placed on a table 230 as shown, or may be placed on the floor, or on a shelf, or in other convenient location. Exemplary positioning of furniture, work surfaces, and doors are indicated in the figure.

FIG. 1C shows a plan view of a sample collection room within a retail location, having a waiting room 120, a sample collection room 110, a transport container 260, a refrigerator 200, and a bathroom 130. In embodiments, a transport container 260 may be placed on the floor as shown, or may be placed on a table 230, or on a shelf, or in other convenient location suitable for supporting or holding a transport container 260. Exemplary positioning of furniture, work surfaces, and doors are indicated in the figure. Other elements shown are labeled as in FIGS. 1A and 1B.

FIG. 1D shows a plan view of a sample collection room within a retail location, having a waiting room 120, a sample collection room 110, a centrifuge 240, a clean refrigerator 204, a biohazard refrigerator 206, a transport container 260, and a bathroom 130. As illustrated in FIG. 1D, in embodiments, a sample collection room 110 may include more than one refrigerator 200; for example, a sample collection room 110 may include two refrigerators, one of which may be a clean refrigerator, and one of which may be a biohazard refrigerator. In embodiments, a sample collection room 110 may include only a clean refrigerator 204 or only multiple clean refrigerators 204; or a sample collection room 110 may include only a biohazard refrigerator 206 or only multiple biohazard refrigerators 206; or may include any combination of these. In embodiments, a biohazard refrigerator 206 may be placed within a cabinet (which is preferably a ventilated cabinet). In embodiments, a biohazard refrigerator 206 may be concealed in a cabinet. In embodiments, a biohazard refrigerator 206 may be located on a table, counter, or shelf, or otherwise (e.g., within a cabinet) be placed at counter height or other height providing convenient access to a technician. In embodiments, a biohazard refrigerator 206 may be located to the right of a surface used to support a target body location for sample collection; e.g., to the right of a warming table, or warming surface, or to the right of a reclining chair, or other furniture, device, or element where sample collection takes place. In embodiments, a clean refrigerator 204 may be placed within a cabinet (which is preferably a ventilated cabinet), and may be concealed in a cabinet. In embodiments, a clean refrigerator 204 may be located on a table, counter, or shelf, or otherwise (e.g., within a cabinet) be placed at counter height or other height providing convenient access to a technician. A clean refrigerator 204 may be used, for example, to store glucose drinks for use in glucose tolerance tests. Exemplary positioning of furniture, work surfaces, and doors are indicated in the figure. Other elements shown are labeled as in FIGS. 1A, 1B, and 1C.

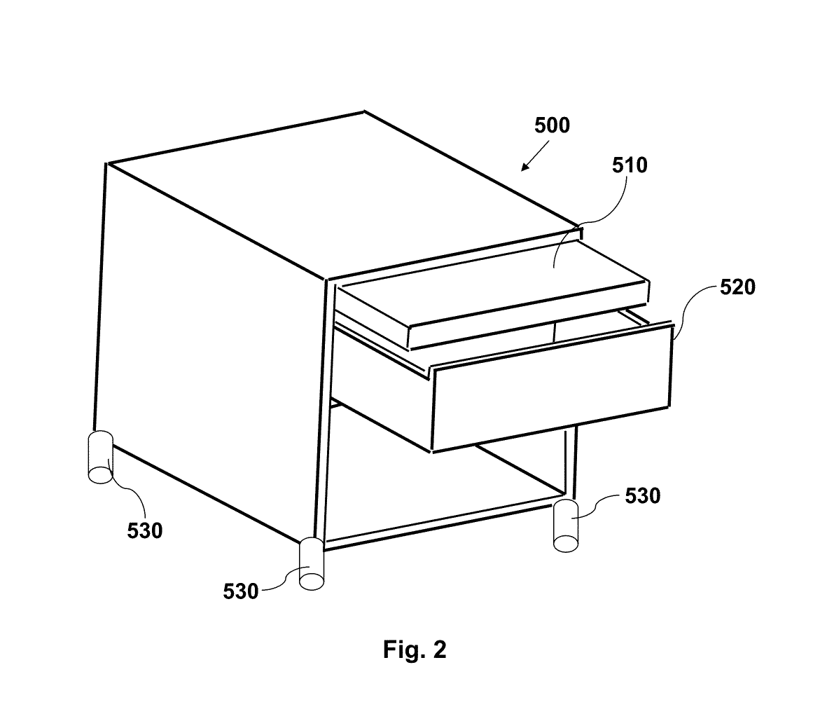

FIG. 2 shows an example of a warming table having a warming surface. The warming table 500 has a warming surface 510 which may be placed in any of several desired locations; as shown, warming surface 510 may slide in and out of a slot. Warming surface 510 also provides a support surface for a body part of a subject, such as an arm or hand, for use during sample collection. Part or all of warming surface 510 may be warmed. Warming table 500 has storage drawers 520 as shown, and adjustable legs 530. The height of adjustable legs 530 may be altered independently of one another, to provide tilt; or may be adjusted together to provide a level surface, as desired.

FIG. 3 shows an example of a warming table including an arm rest as well as a warming surface. Warming table 600 has an extendable arm rest 610 which may be disposed adjacent warming surface 620 for the comfort and convenience of a subject and for more effective sample collection. Warming table 600 has storage drawers 630 as shown, and adjustable legs 640. The height of adjustable legs 640 may be altered independently of one another, to provide tilt; or may be adjusted together to provide a level surface, as desired.

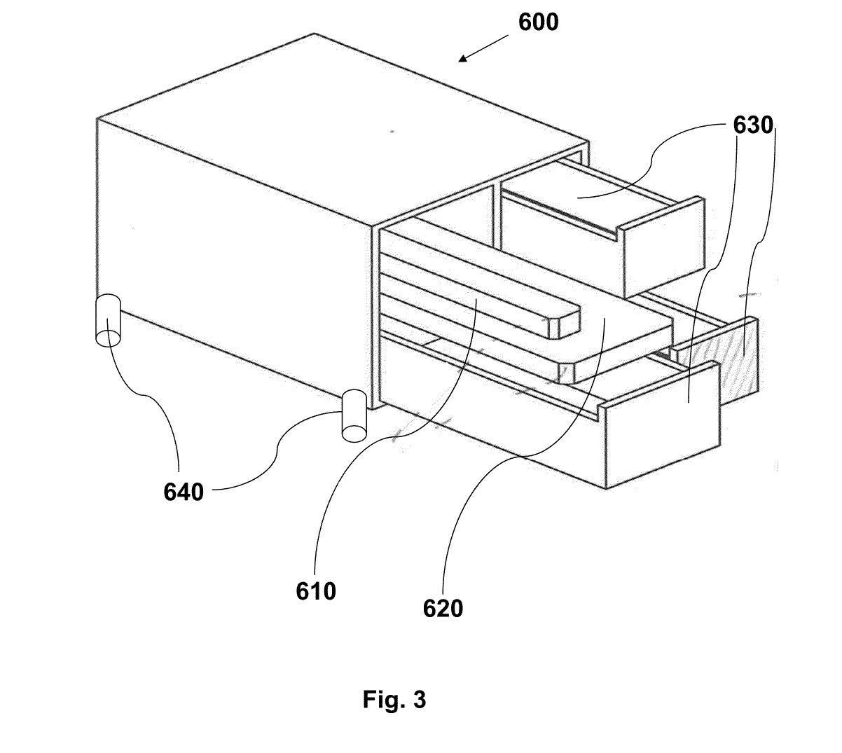

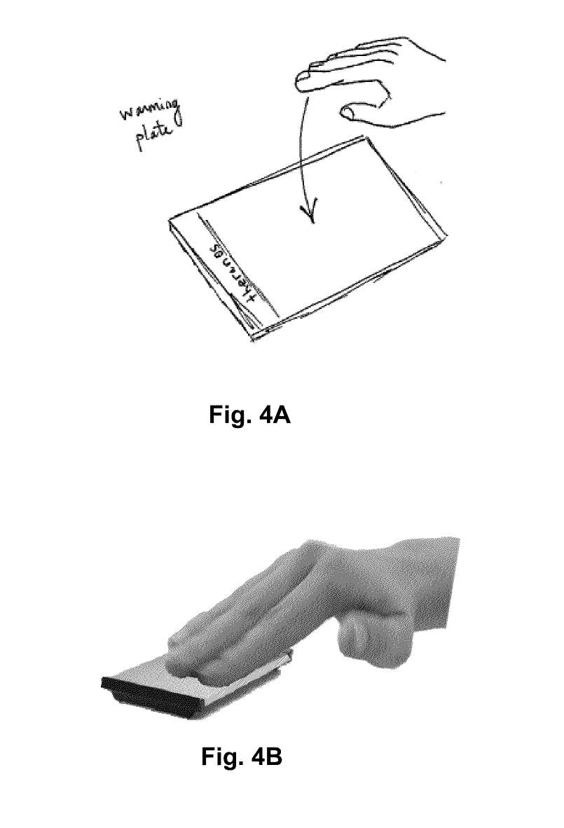

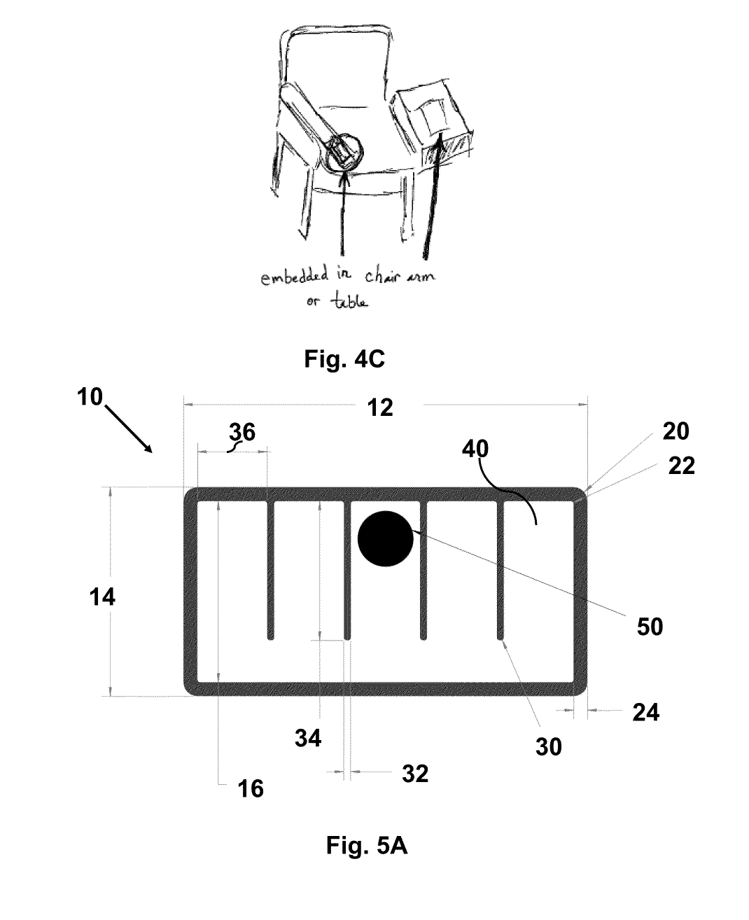

Applicants further disclose a warming plate, which includes a warming element and a surface for contacting a finger, or fingers, or a hand of a subject. In embodiments, the upper surface of a warming plate, or a portion thereof, may be configured for easy cleaning and for maintaining sterility during sample collection. The upper surface of a warming plate may be padded, or include a padded portion. The upper surface of a warming plate may be water-proof, or water-resistant, or include a water-proof, or water-resistant portion. Examples of a warming plate having features as disclosed herein are shown in FIG. 4.

A warming plate as disclosed herein may be a flat plate, suitable for resting on a table, or desk, or shelf, or other surface. A warming plate as disclosed herein may be a flat plate embedded in a table, or desk, or shelf, or other surface. A warming plate as disclosed herein may be a flat plate embedded in the arm of a chair, or an arm of a couch, or an arm-rest attached to, or placed near, a bed or other piece of furniture for seating or resting, positioned in such a way as to allow a subject to rest a finger, or fingers, or hand, or other body part for warming.

The surface of a warming plate may have one or more thermal controlled sites wherein the temperature of the target site on the patient (e.g., a finger) may be brought to a desired temperature. The temperature may be adjustable. By way of example and not limitation, embodiments may heat a finger or other target site to improve blood flow and thus blood yield from a finger-stick. For example, the warming plate may have thermal control areas to increase blood flow to the target area and thus increase the speed with which sufficient blood or other bodily fluid can be drawn from the subject. The heating derived from the heating element of a warming plate is used to bring the target tissue to between about 40° C. to about 50° C. In embodiments, the heating derived from the heating element is configured to bring target tissue to within a temperature range of between about 40° C. to about 44° C. In embodiments, the heating derived from the heating element brings target tissue to within a temperature range of between about 41° C. to about 43° C. In embodiments, the heating derived from the heating element brings target tissue to a temperature of about 42° C. In embodiments, the heating derived from the heating element brings target tissue to within a temperature range of between about 44° C. to about 47° C. In one embodiment, the temperature is sufficient to increase blood flow to yield about 120 uL of sample. In one embodiment, the temperature is sufficient to increase blood flow to yield about 130 uL of sample. In one embodiment, the temperature is sufficient to increase blood flow to yield about 140 uL of sample. In one embodiment, the temperature is sufficient to increase blood flow to yield about 150 uL of sample. Optionally, the thermal controlled site is a shaped surface is contoured to match that of the target site on a patient.

In embodiments, the thermal controlled surface of a warming plate comprises a hard surface that is easily cleaned, and that may be sterilized. Such a surface may be or include, for example, plastic, glass, hard rubber, acrylic, polymer, and other materials. In embodiments, the thermal controlled site comprises a soft or padded surface that is easily cleaned, and that may be sterilized. Such a surface may be or include, for example, cloth, rubberized cloth, soft rubber, and other materials.

FIG. 4A shows a schematic view of an exemplary warming plate, the arrow illustrating the motion to be taken by the hand in order to place one or more fingertips on the surface of the warming plate. Placement of a fingertip, or of more than one fingertip, onto the surface of the warming plate allows warming of the fingertip(s) and aids in increasing blood flow from a subsequent puncture of the fingertip(s), thus aiding collection of a blood sample form the fingertip(s). When in use, the surface of the warming plate is typically at a higher temperature than ambient air temperature, and is effective to warm a fingertip placed thereon to between about 30° C. to about 46° C., or to between about 32° C. to about 45° C., or to between about 34° C. to about 44° C., or to between about 36° C. to about 43° C., or to between about 38° C. to about 42° C., or to between about 39° C. to about 41° C. In embodiments, the surface of a warming plate is maintained during use at a temperature of between about 30° C. to about 46° C., or of between about 32° C. to about 45° C., or of between about 34° C. to about 44° C., or of between about 36° C. to about 43° C., or of between about 38° C. to about 42° C., or of between about 39° C. to about 41° C.

FIG. 4B shows a top view of an exemplary warming plate on which some fingertips of a subject are resting. As shown, the subject's fingertips are being warmed by contact with the warming plate and are thus are being prepared for obtaining a finger-stick blood sample. A slightly different view of a warming plate in use warming fingers of a hand of a subject is shown in FIG. 4C. FIG. 4C illustrates a warming plate placed in the arm of a chair (within the circle in the figure) or on the surface of a cabinet adjacent a chair (arrow in the figure points to a warming plate on the upper surface of a cabinet attached to the chair). In embodiments, a warming plate may be placed on, may be secured to, and may be embedded in, a table, desk, shelf, or other surface. In embodiments, a warming plate may be placed on, may be secured to, and may be embedded in, a surface effective to provide a convenient place for a subject to rest a finger, or fingers, or a hand, during warming. In embodiments, a warming plate may be placed on, may be secured to, and embedded in, the arm of a chair, sofa, couch, or other piece of furniture for seating effective to provide a convenient place for a subject to rest a finger, or fingers, or a hand, during warming.

An example of a chair including a warming plate in an arm rest and a warming plate in a cabinet attached to the chair is shown in FIG. 4C. It will be understood that other seats (e.g., couch, bench, day-bed, or other pieces of furniture for seating, e.g., suitable to seat a subject) may also include a warming plate in an arm rest, similar to the warming plate in a chair arm rest shown in FIG. 4C. The attached cabinet shown in FIG. 4C includes a warming plate; such a cabinet may also be used to store supplies (e.g., supplies for obtaining blood samples, including lancets, containers, swabs, alcohol or other cleaners, band-aids, etc.), equipment, literature, and other utensils and material related to the collection of blood or other biological samples from a subject. In embodiments, a cabinet with a warming plate may be placed adjacent to, or near, a chair, without being attached to the chair.

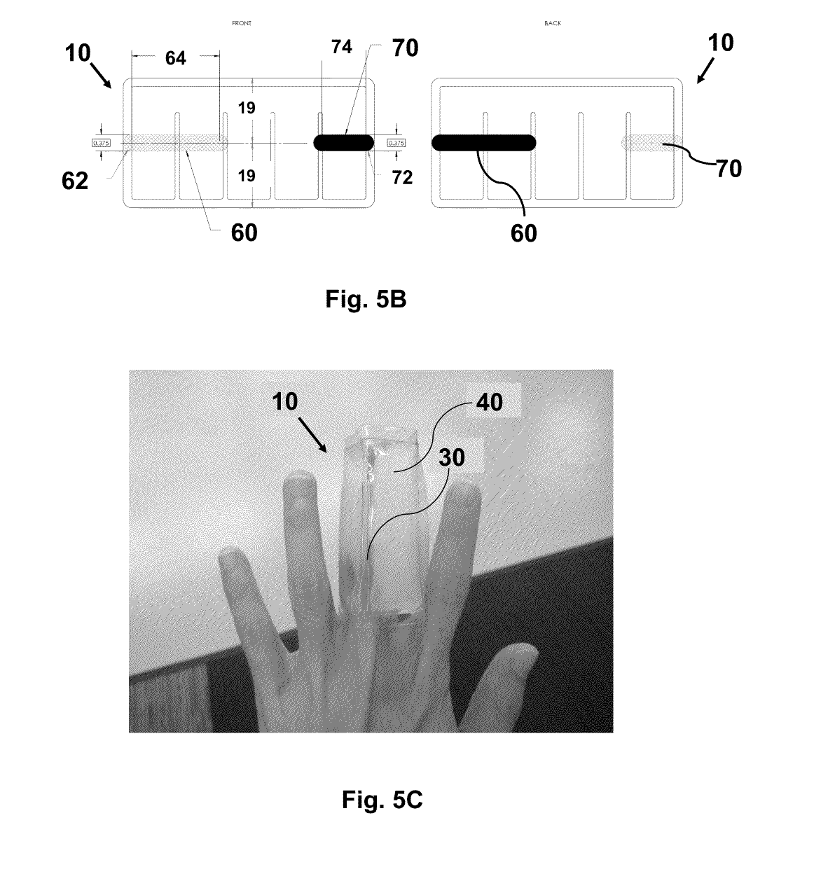

FIG. 5 shows examples of a finger warmer which may be used to warm a subject's finger (or other extremity, e.g., a toe, or a heel) in order to enhance blood flow from the extremity during acquisition of a biological sample from the subject. A finger warmer having features as illustrated in FIGS. 5A, 5B, and 5C may be, for example, activated to provide warmth and may be wrapped around a finger (or two or more fingers). The sample may be acquired after a minute, or two minutes, or three minutes, or more after wrapping. For example, following a few minutes of warming, the finger or fingers of the subject may be unwrapped sufficiently to expose a fingertip, and a lancet applied so as to pierce the skin and allow a few droplets of blood to escape the fingertip and be collected as a biological sample.

FIG. 5A shows a schematic view of an example of a finger warmer having features as disclosed herein, providing a top view of a finger warmer illustrating some design features and exemplary dimensions. FIG. 5B shows a schematic example of a finger warmer having features as disclosed herein, providing a front view of a finger warmer and a back view of a finger warmer. A finger warmer having features as disclosed herein comprises a closed container enclosing a heating composition which can be triggered to provide heat. Thus, a finger warmer may comprise an enclosed compartment, having a flexible outer covering (e.g., a “skin”), such as a polymer, plastic, rubber, cloth, or other flexible material, enclosing materials which may, when desired, react in an exothermic reaction effective to release heat which may be used to warm an extremity of a subject. The flexible outer covering, or an inner portion of such a covering, is typically suitable for containing the enclosed materials without leakage, and is typically suitable for containing the enclosed materials in a state which prevents the occurrence of the exothermic reaction until it is purposefully triggered. Such an outer covering may be a water-proof covering, may be an air-tight covering, and, in embodiments, may be both water-proof and air-tight.

An outer covering of a finger warmer as disclosed herein is typically soft, or flexible, or both, and is typically configured to allow bending, folding, or other manipulation effective, e.g., to wrap a finger warmer around a finger, or around two or more fingers, of a subject. In embodiments, an outer covering of a finger warmer may comprise a polymer, such as Nylon®, Dacron®, Perlon®, Terylene®, Kevlar®, and other polymers or polymer blends. In embodiments, an outer covering of a finger warmer may comprise polyvinylchloride, polyethylene, polyurethane, or other polymer. In embodiments, an outer covering of a finger warmer may comprise a metallocene polymer, or a metallocene blend, such as, e.g., a metallocene nylon blend. In embodiments, a finger warmer may comprise a metallocene blend/nylon/metallocene blend composite. The perimeter of the outer covering, and the regions shown as vertical lines within the perimeter in FIGS. 5A and 5B, may be fused together, e.g., by heat, or glue, or ultrasonic welding, or other means. In embodiments, a finger warmer may comprise an internal baffle, or a plurality of internal baffles, or may comprise two or more enclosed compartments. In embodiments, a finger warmer may be foldable, e.g., may be folded around an extremity of a subject, or folded around itself (folded up, as for storage). In embodiments, a finger warmer may be configured to be rolled, e.g., may be rolled around an extremity of a subject, or rolled around itself (e.g., rolled up, as for storage). In embodiments, a finger warmer may be flexible. In embodiments, a finger warmer may comprise hinges. As shown in FIG. 5A in an extended configuration, the finger warmer may have a substantially rectangular shape; the vertical lines within the rectangular perimeter indicate positions configured for folding the finger warmer. In embodiments, a finger warmer in an extended configuration may have a square, or round, or oval, or other shape.

As shown in FIG. 5A and FIG. 5B, a finger warmer 10 may have a perimeter 20 that may be roughly rectangular in shape, with a length 12 of between about 3 to about 12 inches, or between about 4 to about 10 inches, or between about 4.5 and about 8 inches, or between about 5 and about 7 inches. A perimeter 20 may enclose an inner perimeter 22. In embodiments, a finger warmer 10 may have a length 12 of about 6 inches. A finger warmer 10 may have a width 14 of between about 1 and about 6 inches, or between about 2 and about 5 inches, or between about 2.5 and about 4 inches. In embodiments, a finger warmer 10 may have a width 14 of about 3 inches. A finger warmer 10 may include an outer seam, or weld, which may have a seam width 24 of between about 0.05 inches to about 0.5 inches, or between about 0.1 inches to about 0.3 inches, or of about 0.2 inches. A finger warmer 10 may thus provide an inner compartment 40, within the outer seam 24, having an internal width 16 of between about 0.5 and about 5 inches, or between about 1 and about 4 inches, or between about 1.5 and about 3 inches, or of about 2.5 inches.

A finger warmer 10 may include an internal baffle 30 or multiple internal baffles 30, which may facilitate folding a finger warmer by providing a thinner, or lower-resistance portion than other portions of the finger warmer 10. Internal baffles 30 may extend between one wall of the perimeter 20 and an opposite wall, or, as shown in FIGS. 5A and 5B, an internal baffle 30 may extend partially from one wall, stopping short of the opposite wall of perimeter 20. A first internal baffle 30 may be spaced apart from a second internal baffle 30, for example, by a spacing 36 of between about 0.3 to about 2 inches, or of between about 0.5 to about 1.5 inches, or of about 1 inch. An internal baffle 30 may have a baffle width 32 of between about 0.01 to about 0.5 inches, or between about 0.05 to about 0.2 inches, or may have a baffle width 32 of about 0.1 inches. An internal baffle 30 may have a baffle length 34 of between about 0.5 to about 6 inches, or may have a baffle length 34 of between about 1 to about 4 inches, or between about 1.5 to about 3 inches. In embodiments, an internal baffle 30 may have a baffle length 34 of about 2 inches.

A finger warmer 10 provides an enclosed space (e.g., internal compartment 40 shown in FIGS. 5A and 5B). In embodiments, an internal compartment 40 may comprise a volume of between about 5 mL to about 250 mL; or between about 10 mL and about 200 mL; or between about 15 mL and about 100 mL; or between about 20 mL and about 80 mL; or between about 30 mL and about 60 mL; or between about 35 mL and about 45 mL.

A finger warmer 10 as shown in FIGS. 5A and 5B may have a single internal compartment 40 (since, in this illustration, baffle length 34 is less than internal width 16, so that internal baffles 30 do not completely isolate individual portions of internal compartment 40). In embodiments, a finger warmer 10 may provide a plurality of enclosed spaces (e.g., enclosed spaces separated by barriers similar to internal baffles 30 but which extend completely from one perimeter wall to another, closing off one portion of an internal compartment 40 from another portion of an internal compartment 40).

An enclosed space (e.g., internal compartment 40 shown in FIGS. 5A and 5B) of a finger warmer 10 may hold a heating composition (e.g., a composition capable of providing heat, by, for example, an exothermic reaction). For example, the enclosed space of a finger warmer may hold a supersaturated salt solution (e.g., supersaturated sodium acetate in water) containing a trigger element, such as a metal disk which, when flexed, triggers crystallization of the salt, thereby releasing heat. In embodiments, the enclosed space of a finger warmer may hold a water-based solution including iron (typically as a powder), typically with a salt, and often also with activated carbon and other materials; the iron, when exposed to air, releases heat as the iron is oxidized, effective to warm a subject or a subject's extremity.

A finger warmer 10 may contain an oxidizable composition (e.g., a composition containing iron particles); or other composition which may release heat under proper conditions. An oxidizable composition may be contained or stored so as to prevent contact between the composition and air (or more particularly, between the composition and oxygen in the air); providing access to air initiates the desired heat generation. Thus, a finger warmer 10 containing an oxidizable composition within an internal compartment 40 may be maintained ready for use by preventing contact between the oxidizable composition and air, and heating may be initiated by allowing contact between the oxidizable composition and air (e.g., by removing a covering over a port which allows contact between the oxidizable composition and air, or otherwise breaching an air-tight enclosure containing the oxidizable composition).

A finger warmer 10 may contain a super-saturated solution, such as a super-saturated salt solution, which may release heat under proper conditions. A super-saturated salt solution (such as super-saturated sodium acetate) may include therein a trigger 50. A trigger 50 may be, for example, a metal disk which upon deformation is able to trigger the release of heat from the super-saturated solution. In embodiments in which a finger warmer 10 has a single internal compartment 40, activation of heating composition within the internal compartment 40 eventually activates all of the heating composition. In embodiments, such as embodiments in which an internal baffle 30, or multiple internal baffles 30, extend from one wall of perimeter 20 to an opposite wall of perimeter 20 to isolate one portion of internal compartment 40 from another portion of internal compartment 40, a finger warmer 10 may have separate portions of a heating composition enclosed in separate internal compartments 40. In such embodiments, in which a finger warmer 10 has multiple internal compartments 40, activation of heating composition within one internal compartment 40 may not activate the heating composition in another internal compartment 40, or may not activate the heating composition in all of the internal compartments 40.

In FIG. 5A, a trigger 50 is shown in place within the internal compartment 40. A trigger 50 is used to initiate the release of heat from a heating composition within a finger warmer 10 (such initiation may be termed “activation” of the heating composition). For example, flexing or deformation of a trigger 50 within a super-saturated salt solution may be effective to initiate salt crystallization in the super-saturated solution; when the salt is an appropriate salt, such crystallization generates heat. For example, deformation of a trigger 50 within a super-saturated sodium acetate solution is able to trigger crystal formation in that solution. Without being bound by theory, it is believed that sodium acetate crystals formed in cracks, on peaks or discontinuities, or on the surface of the trigger 50 may be dislodged by deformation of the trigger 50, and so made available as nucleation centers for further sodium acetate crystal formation. In embodiments, a trigger 50 may be a packet or container holding sodium acetate (or other salt) crystals, and be able to initiate release of heat from a surrounding super-saturated sodium acetate (or other salt) solution by release of the crystals or other contact between the crystals and the super-saturated solution (e.g., by tearing or otherwise opening the packet or container).

In embodiments, a trigger 50 may comprise a metal disk, e.g., a thin metal disk having a thickness of between about 0.05 to about 0.4 inches, or between about 0.1 to about 0.3 inches, or of about 0.2 inches. In embodiments, a trigger 50 may comprise a thin metal disk having a diameter of between about 0.1 to about 2 inches, or of between about 0.3 to about 1.5 inches, or of between about 0.5 to about 1 inches, or of about 0.75 inches. In embodiments, a trigger 50 is typically made of a material which does not degrade or decay when in contact with sodium acetate or other salt. In embodiments a trigger 50 may be a stainless steel disk; in alternative embodiments, a trigger 50 may be made of another metal, or other material able to trigger an exothermic reaction when flexed or broken. In embodiments, a trigger 50 may have a square, or rectangular, or oval, or other shape. In embodiments, a trigger 50 may have a continuous surface, or may have holes, or pores, or other features breaking up the continuity of the trigger surface. In embodiments, a trigger 50 may have a flat surface, or may have an irregular surface, or a rough surface, or may include grooves, depressions, ridges, elevations, or other features rising from, or dropping into, the surface of a trigger 50.

Prior to activation of a heating composition in the internal compartment 40 of a finger warmer 10, the finger warmer 10 and its contents are typically at room temperature. Following activation of a heating composition, the finger warmer 10 and its contents may reach temperatures of between about 32° C. to about 48° C., or between about 35° C. to about 45° C., or between about 37° C. to about 43° C., or between about 39° C. to about 41° C. Heat generation to these levels may last for about 5 to 15 minutes, or about 8 to 10 minutes, following initiation of the heating process. If desired, the temperature range may be increased by alteration of the heating composition (e.g., by increasing the concentration of sodium acetate in a super-saturated sodium acetate solution, or by increasing the amount of concentration of iron in heating composition that relies on oxidation of iron to produce heat, etc.). Alternatively, lowering the concentration of salt or iron, or other active component of the heating composition may reduce the temperature range if less heat generation is desired.

FIG. 5B shows both the front side (left-hand image) and back side (right-hand image) of a finger warmer 10 having features disclosed herein. A finger warmer 10 may have fasteners 60 and 70. Fasteners 60 and 70 may be used to secure a finger warmer 10 when the finger warmer 10 is rolled or folded around a finger. Fasteners 60 and 70 may be, for example, Velcro® fasteners which, when in contact with each other, are able to hold a finger warmer 10 in a rolled or folded configuration around a finger of a subject. Fasteners 60 and 70 may comprise different, complementary, fastener elements; for example where fasteners 60 and 70 comprise Velcro® fasteners, an external surface of fastener 60 differs from the corresponding, and complementary, surface of fastener 70. A further example in which fasteners 60 and 70 comprise different fasteners is found where fasteners 60 and 70 form a snap, in which one of fasteners 60 and 70 has a protruding portion which fits snugly into a depression or receptacle in the other of fasteners 60 and 70. In embodiments, fastener 60 and fastener 70 may be similar, or may be identical; for example fastener 60 and fastener 70 may both comprise hooks configured to latch onto each other, or may comprise two portions of a zipper, or may comprise other fasteners. A fastener 60 may have a fastener width 62 and a fastener length 64; a fastener 70 may have a fastener width 72 and a fastener length 74. In the example shown in FIG. 5B, fastener width 62 and fastener length 64 are different than fastener width 72 and fastener length 74; however, in embodiments, fastener width 62 and fastener length 64 may be the same as fastener width 72 and fastener length 74. In embodiments, as shown in FIG. 5B, fasteners 60 and 70 may be placed along a midline (e.g., with centers along a line that is a distance 19 from an outer edge of perimeter 20); placement along a midline as shown facilitates opposition of fastener 60 with fastener 70 and aids in their function of fastening to each other, effective to hold a finger warmer 10 in a fastened configuration. In embodiments, so long as they may be manipulated so as to make contact and to fasten with each other, fasteners 60 and 70 may be placed asymmetrically. In embodiments, a finger warmer 10 may have a strap, or belt, which may be used to hold a rolled or folded finger warmer 10 in a rolled or folded configuration, e.g., around a finger.

FIG. 5C shows a finger warmer 10 wrapped around the finger of a subject. The finger warmer 10 has been folded along baffles 30, and, as it is flexible, is also curved or flexed in other locations as well. Such folds, and such curvature, whether alone, or whether together, aid in wrapping a finger warmer 10 around a finger, or portion thereof, of a subject, and aid in warming the finger, or portion thereof, of the subject in order to facilitate acquisition of a biological sample from the subject.

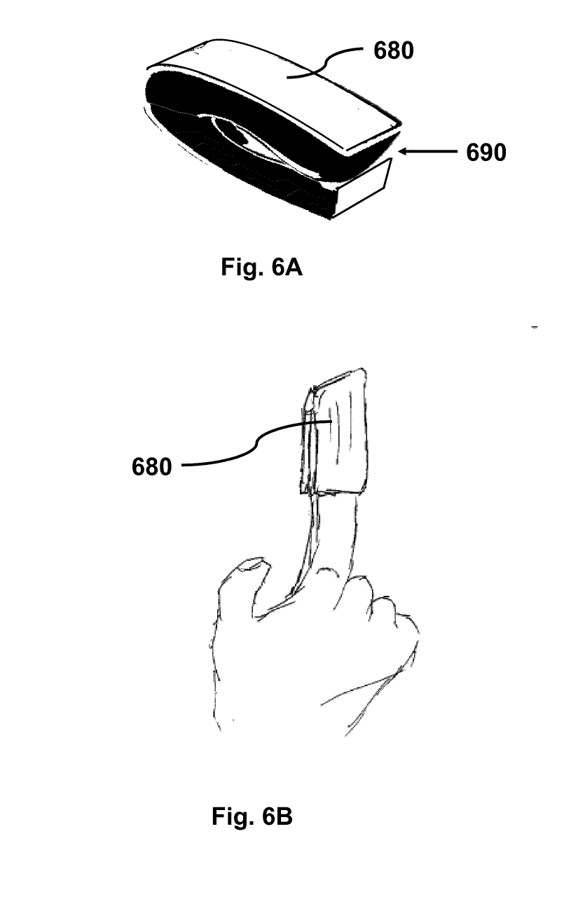

FIG. 6 shows an example of a fingertip warmer having features as disclosed herein. As shown, a fingertip warmer may be a portable device which can enclose at least part of a finger, or at least part of a fingertip, in preparation for obtaining a blood sample from a finger of a subject. A side perspective view of an exemplary fingertip warmer 680 is shown in FIG. 6A. A fingertip may be inserted into a fingertip warmer 680 via slot 690. A fingertip warmer 680 is shown in place around the fingertip of a subject in FIG. 6B. A fingertip warmer may include a heating element, or may be connected to a heating element, effective to warm a fingertip. The results of warming due to a fingertip warmer may be substantially the same as the warming from a warming table, or from a warming plate; that is, for example, the fingertip may be warmed to a temperature of between about 32° C. to about 48° C. For example, a fingertip warmer may be effective to warm a fingertip of a subject to between about may reach temperatures of between about 32° C. to about 48° C., or between about 35° C. to about 45° C., or between about 37° C. to about 43° C., or to between about 39° C. to about 41° C. In embodiments, a fingertip warmer may be effective to warm a fingertip of a subject to a temperature of between about 39° C. to about 40° C. In embodiments, a fingertip warmer may be effective to warm a fingertip of a subject to a temperature of about 38° C. In embodiments, a fingertip warmer may be effective to warm a fingertip of a subject to a temperature of about 39° C. In embodiments, a fingertip warmer may be effective to warm a fingertip of a subject to a temperature of about 40° C. In embodiments, a fingertip warmer may be effective to warm a fingertip of a subject to a temperature of about 41° C. In embodiments, a fingertip warmer may be effective to warm a fingertip of a subject to a temperature of about 42° C. Such warming by a fingertip warmer may be sufficient, for example, to enhance blood flow from a finger-stick or other puncture to yield about 100 uL of sample, about 110 uL of sample, or about 120 uL of sample, or about 130 uL of sample, or about 140 uL of sample, or about 150 uL of sample, or more.

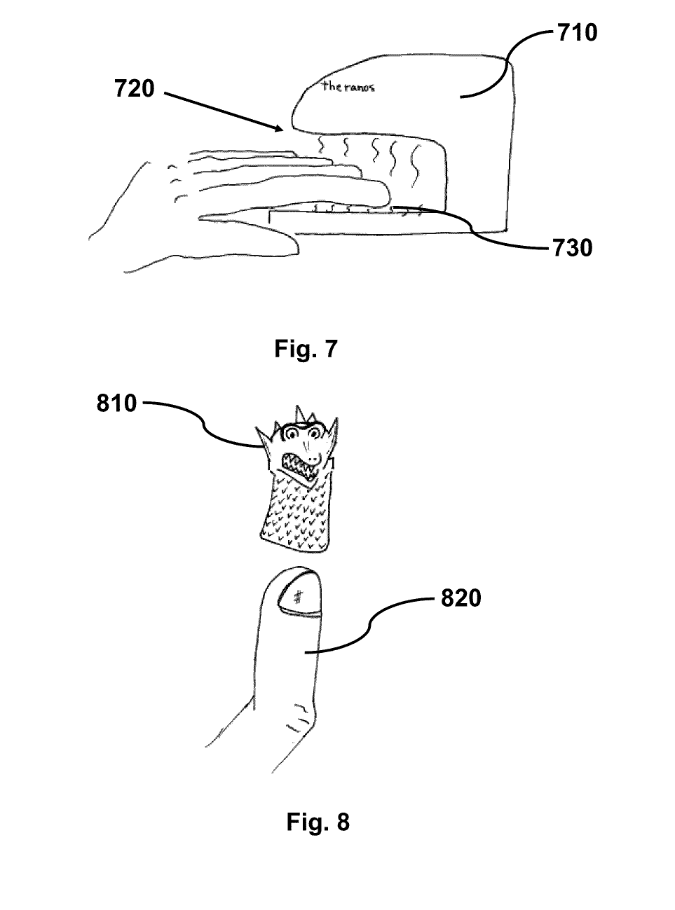

A further embodiment of devices for warming a fingertip, or for warming a plurality of fingers, or for warming a hand of a subject is shown in FIG. 7. The example shown in FIG. 7 illustrates such a warmer which utilizes warm air to warm a fingertip, or finger, or fingers, or a hand. A hand may be inserted into aperture 720 of a warmer 710 as illustrated in FIG. 7. A warmer 710 as illustrated in FIG. 7 may also include a surface 730 for resting a fingertip, finger, fingers, or hand of a subject; in embodiments, such a surface may be configured to guide or place a fingertip, finger, fingers, or hand of a subject into a proper or desired position or orientation for warming. As shown, the warmer 710 may be configured to at least partially enclose a hand, or fingers, or a subject. In embodiments, a warmer 710 as illustrated in FIG. 7 may include a heating element and a fan. In embodiments, a warmer 710 as illustrated in FIG. 7 may provide heating via convection, or other means of circulating air. a warmer 710 as illustrated in FIG. 7 may include a source of radiant heat, such as, e.g., a heat lamp or other infrared light source. In embodiments, a warmer using warm air to warm a finger or a fingertip may be configured to enclose a single finger, or to enclose a portion of a single finger (e.g., a fingertip) and to direct warm air towards, and onto, the fingertip, finger, fingers, or hand of the subject. A warmer 710 having features a s disclosed herein may also include a screen, or a baffle, or vanes, or other elements to direct the flow of air in the desired direction. In addition, a screen, or a baffle, or vanes, or other elements may be effective to insure that the fingertip, or finger, or fingers, or hand of the subject does not approach too closely to the source of air (e.g., an orifice or vent); or to a fan or fan blades; or to a heating element; or other element or portion of the air-warmer to which contact with a subject is not desired.

Applicants disclose herein fingertoys which may fit onto a fingertip, and which may serve as finger warmers, or as bandages, or to provide compression, or for other purposes. For example, placement of a fingertoy on a fingertip of a child may provide comfort, or amusement, or diversion, to the child. A fingertoy may be placed on the finger of a child or other subject prior to acquisition of a blood sample from a fingertip. A fingertoy may be placed on the finger of a child or other subject after acquisition of a blood sample from a fingertip. A fingertoy may be placed on the finger of a child or other subject prior to acquisition of a blood sample from a fingertip, and may be again placed on the finger of a child or other subject following acquisition of a blood sample from a fingertip. Thus, a subject, such as a child, may don a fingertoy prior to, or following puncture of a finger (e.g., following a fingerstick), for comfort, warmth, and amusement.

An example of such a fingertoy is shown in FIG. 8. A fingertoy 810 may be placed over a portion of the finger 820, such as a fingertip; for example, a fingertoy 810 as disclosed herein may fit over a fingertip of a finger 820 in a manner similar that is similar to the manner in which a thimble is commonly placed over a fingertip (or thumb-tip) of a person seeking to protect a fingertip during sewing. In embodiments, a fingertoy 810 may be placed over a substantial portion of a finger 820, or over all of a finger 820, in a manner similar to the manner in which the finger of a glove covers a finger. A fingertoy 810 may be made from, or may include (e.g., as an inner lining) an absorbent material. A fingertoy 810 may be used in place of, or in conjunction with, a bandaid or other bandage. A fingertoy 810 may include perfume or other odorant. A fingertoy 810 may keep a finger warm, or may provide compression, or may provide other physical sensations which may act to alleviate any discomfort which may ensue following a fingerstick.

As shown, fingertoy 810 may include features which may suggest a face, or arms, or hair, or other features which may be amusing, or diverting, or comforting, to a subject (e.g., a child) who has had a blood sample taken from a finger. A fingertoy 810 may include writing, or symbols, or other markings which may provide amusement, diversion, or comfort, or which convey a message or reminder to a subject. In embodiments, a fingertoy 810 may identify the location or enterprise which performed or collected the sample. In embodiments, a fingertoy 810 may include a commercial message, or may include symbols or other markings which convey a commercial message.

Methods for obtaining a sample from a subject may comprise: providing a sample collection room in a retail store, the sample collection room including a warming table; and collecting a sample from a subject. Methods for obtaining a sample from a subject may comprise: providing a sample collection room in a retail store, the sample collection room including a warming plate; and collecting a sample from a subject. Applicants disclose methods for obtaining a sample from a subject, comprising providing a sample collection room in a retail store, the sample collection room including a warming plate; warming the subject's fingertip, or finger, or hand, or heel, or toe-tip, or toe (e.g., using the warming table), and collecting a sample from a subject. In embodiments, the sample is a blood sample, e.g., a blood sample obtained from a fingerstick.

Methods for obtaining a sample from a subject may comprise: providing a sample collection room in a retail store, the sample collection room including a chair, bench, couch, or other piece of furniture for seating having a warming plate or other warming element (e.g., in an armrest, on a cabinet, or other surface conveniently placed near to the subject); and collecting a sample from a subject. Applicants disclose methods for obtaining a sample from a subject, comprising providing a sample collection room in a retail store, the sample collection room including a chair, bench, couch, or other piece of furniture for seating having a warming plate or other warming element (e.g., in an armrest, on a cabinet, or other surface conveniently placed near to the subject); warming the subject's fingertip, or finger, or hand, or heel, or toe-tip, or toe (e.g., using the warming plate or other warming element), and collecting a sample from a subject. In embodiments, the sample is a blood sample, e.g., a blood sample obtained from a fingerstick.

Methods for obtaining a sample from a subject may comprise: providing a sample collection room in a retail store, the sample collection room including an air-warmer; and collecting a sample from a subject. Applicants disclose methods for obtaining a sample from a subject, comprising providing a sample collection room in a retail store, the sample collection room including an air-warmer; warming the subject's fingertip, or finger, or hand, or heel, or toe-tip, or toe (e.g., using the air-warmer), and collecting a sample from a subject. In embodiments, the sample is a blood sample, e.g., a blood sample obtained from a fingerstick.

Methods for obtaining a sample from a subject may comprise: providing a sample collection room in a retail store, the sample collection room including a finger warmer; and collecting a sample from a subject. Applicants disclose methods for obtaining a sample from a subject, comprising providing a sample collection room in a retail store, the sample collection room including a finger warmer; warming the subject's fingertip, or finger, or hand, or heel, or toe-tip, or toe (e.g., using the finger warmer), and collecting a sample from a subject. In embodiments, the sample is a blood sample, e.g., a blood sample obtained from a fingerstick.

Methods for obtaining blood from a subject in a sample collection room may further include providing a post-draw toy to the subject (e.g., a child subject). For example, methods include obtaining blood from a subject in a sample collection room, using a warming plate and optionally providing a post-draw toy to the subject (e.g., a child subject), such as, e.g., a fingertoy. Methods may include obtaining blood from a subject in a sample collection room, using a warming plate (e.g., as illustrated in FIG. 4A, 4B, or 4C), and optionally providing a post-draw toy to the subject (e.g., a child subject). Methods may include obtaining blood from a subject in a sample collection room, using a finger-warmer (e.g., as illustrated in FIG. 5A, 5B, or 5C) and optionally providing a post-draw toy to the subject (e.g., a child subject). Methods may include obtaining blood from a subject in a sample collection room, using an air-warmer (e.g., an air-warmer as illustrated in FIG. 6A or 6B) and optionally providing a post-draw toy to the subject (e.g., a child subject).

Applicants disclose methods for analyzing a sample obtained from a subject, comprising providing a sample collection room in a retail store; collecting a sample from a subject; and analyzing the sample. In embodiments, the sample collection room includes one or more of a warming table; a chair or other piece of furniture for seating having a warming plate or other warming element; an air-warmer; a calming feature; a finger warmer; a fingertoy; and other elements. In embodiments, the sample collection room is adjacent to a bathroom. In embodiments, the sample collection room comprises a pass-through connecting to a bathroom adjacent to the sample collection room. In embodiments, the sample is analyzed at, or adjacent to, or near to, the sample collection room in the retail store. In embodiments, the sample is analyzed at a location other than at the retail store.

Accordingly, Applicants disclose methods for obtaining and analyzing a sample obtained from a subject, comprising: providing a sample collection room in a retail store; collecting a sample from a subject in the sample collection room; and analyzing the sample. Applicants disclose methods for obtaining and analyzing a sample obtained from a subject, comprising: providing a sample collection room in a retail store; collecting a small sample from a subject in the sample collection room; and analyzing the small sample. Applicants disclose methods for obtaining and analyzing a sample obtained from a subject, comprising: providing a sample collection room in a retail store; collecting a sample from a subject in the sample collection room; and analyzing the sample at the retail store location, or adjacent to the retail store location. In embodiments, these methods may include warming a body part of a subject prior to, or during, obtaining a sample from the body part. Such warming may include use of a warming table, a warming plate, an air-warmer, a finger warmer, a fingertoy, a chair or other furniture for seating including a warming plate or other warming element, and combinations thereof.

A method for obtaining a sample from a subject, said subject having a body part, comprising: providing a sample collection room in a retail store; and obtaining a sample from said body part of said subject, wherein said obtaining is performed in said sample collection room. In embodiments, the methods comprise warming a body part; for example, a body part may be warmed prior to obtaining a sample from the body part, or may be warmed during obtaining a sample. In embodiments, warming of the body part comprises warming with a warming table. In embodiments, warming of the body part comprises warming with a warming plate. In embodiments, warming of the body part comprises warming with a finger warmer. In embodiments, warming of the body part comprises warming with a fingertoy. In embodiments, warming of the body part comprises warming with an air-warmer. In embodiments, the body part comprises a fingertip, a finger, a plurality of fingers, a hand, or other body part or body parts.

A method for analyzing a sample obtained from a subject, said subject having a body part, comprising: providing a sample collection room in a retail store; obtaining, in said sample collection room, a sample from said body part of said subject; and analyzing the sample at or adjacent to the retail store. In embodiments, the methods comprise warming a body part; for example, a body part may be warmed prior to obtaining a sample from the body part, or may be warmed during obtaining a sample. In embodiments, warming of the body part comprises warming with a warming table. In embodiments, warming of the body part comprises warming with a warming plate. In embodiments, warming of the body part comprises warming with an air-warmer. In embodiments, warming of the body part comprises warming with a finger warmer. In embodiments, warming of the body part comprises warming with a fingertoy. In embodiments, the body part comprises a fingertip, a finger, a plurality of fingers, a hand, or other body part or body parts.

A method for analyzing a sample obtained from a subject, said subject having a body part, comprising: providing a sample collection room in a retail store; obtaining, in said sample collection room, a sample from said body part of said subject; and analyzing the sample at or adjacent to the retail store within a short period of time after obtaining the sample. In embodiments, the methods comprise warming a body part; for example, a body part may be warmed prior to obtaining a sample from the body part, or may be warmed during obtaining a sample. In embodiments, warming of the body part comprises warming with a warming table. In embodiments, warming of the body part comprises warming with a warming plate. In embodiments, warming of the body part comprises warming with an air-warmer. In embodiments, warming of the body part comprises warming with a finger warmer. In embodiments, warming of the body part comprises warming with a fingertoy. In embodiments, the body part comprises a fingertip, a finger, a plurality of fingers, a hand, or other body part or body parts.

A method for analyzing a sample obtained from a subject, said subject having a body part, comprising: providing a sample collection room in a retail store; obtaining, in said sample collection room, a sample from said body part of said subject; transporting the sample to an analysis location; and analyzing the sample at said analysis location within a short period of time after obtaining the sample. In embodiments, the methods comprise warming a body part; for example, a body part may be warmed prior to obtaining a sample from the body part, or may be warmed during obtaining a sample. In embodiments, warming of the body part comprises warming with a warming table. In embodiments, warming of the body part comprises warming with a warming plate. In embodiments, warming of the body part comprises warming with an air-warmer. In embodiments, the body part comprises a fingertip, a finger, a plurality of fingers, a hand, or other body part or body parts.

In embodiments of the methods disclosed herein, a sample may be centrifuged prior to placement in a transport container. In embodiments of the methods disclosed herein, a sample may be centrifuged prior to placement in a sample analysis device. In embodiments of the methods disclosed herein, a sample may be centrifuged as part of sample preparation for analysis in a sample analysis device.

In embodiments of the methods disclosed herein, a sample may be placed in a refrigerator prior to placement in a transport container. In embodiments of the methods disclosed herein, a sample may be placed in a refrigerator prior to placement in a transport container, and may be transported in a refrigerated condition; in embodiments, a transport container may be a refrigerated transport container, effective to transport a sample in a refrigerated condition. In embodiments of the methods disclosed herein, a sample may be placed in a refrigerator prior to placement in a sample analysis device. In embodiments of the methods disclosed herein, a sample may be refrigerated as part of sample preparation for analysis in a sample analysis device.

Further methods disclosed herein include methods for analyzing a sample, comprising collecting a sample in a sample collection room as disclosed herein, and placing the sample in a transport container. In embodiments, a sample placed in a transport container may be transported to a sample analysis location; or may be stored in the transport container at or near the sample collection room for a period of time prior to being transported to a sample analysis location.

A sample obtained by such methods may be a small sample. For example, such a sample may be obtained from a finger-stick, and may comprise a few drops, or two drops, or one drop, of blood obtained from a small lancet puncture in the skin of the subject. The sample, such as a blood sample, may be obtained from the subject following warming a body part (e.g., a fingertip, or finger, of fingers, or hand) of subject. For example, in embodiments, the sample is a small volume sample of blood, or of urine, or of saliva, or of tears, or other bodily secretion or excretion. For example, in embodiments, the sample is a small sample having a volume of less than about 200 μL, or less than about 150 μL, or less than about 100 μL, or less than about 75 μL, or less than about 50 μL, or less than about 25 μL, or less.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

Additionally, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a size range of about 1 nm to about 200 nm should be interpreted to include not only the explicitly recited limits of about 1 nm and about 200 nm, but also to include individual sizes such as 2 nm, 3 nm, 4 nm, and sub-ranges such as 10 nm to 50 nm, 20 nm to 100 nm, etc.

The publications discussed or cited herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. All publications mentioned herein are incorporated herein by reference to disclose and describe the structures and/or methods in connection with which the publications are cited. The following applications are fully incorporated herein by reference for all purposes:

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. Any feature, whether preferred or not, may be combined with any other feature, whether preferred or not. The appended claims are not to be interpreted as including means-plus-function limitations, unless such a limitation is explicitly recited in a given claim using the phrase “means for.” It should be understood that as used in the description herein and throughout the claims that follow, the meaning of “a,” “an,” and “the” includes plural reference unless the context clearly dictates otherwise. For example, a reference to “an assay” may refer to a single assay or multiple assays. Also, as used in the description herein and throughout the claims that follow, the meaning of “in” includes “in” and “on” unless the context clearly dictates otherwise. Finally, as used in the description herein and throughout the claims that follow, the meaning of “or” includes both the conjunctive and disjunctive unless the context expressly dictates otherwise. Thus, the term “or” includes “and/or” unless the context expressly dictates otherwise.

This document contains material subject to copyright protection. The copyright owner (Applicant herein) has no objection to facsimile reproduction of the patent documents and disclosures, as they appear in the US Patent and Trademark Office patent file or records, but otherwise reserves all copyright rights whatsoever. The following notice shall apply: Copyright 2013-2014 Theranos, Inc.

1. A system for obtaining a sample from a subject, comprising a sample collection room in a retail store, said sample collection room comprising a sample transport container configured to hold a plurality of sample containers, said sample containers configured to hold fluid samples having volumes of about 500 μL or less, and one or more of: a warming table, a warming plate, a chair or other furniture for seating comprising a warming plate, a finger warmer, a fingertip warmer, and an air-warmer.

2. A system for analyzing a sample from a subject, comprising a sample collection room in a retail store, said sample collection room comprising a sample analysis device configured to analyze a fluid sample having volumes of about 500 μL or less obtained in said sample collection room, and one or more of: a warming table; a reclining chair configured for positioning a subject effective for obtaining a sample; and a calming feature.

3. The system for analyzing a sample from a subject of claim 2, further comprising a chair or other furniture for seating comprising a warming plate or other heating element, the chair or other furniture for seating being configured to warm a body part of said subject, and being configured for positioning a subject effective for obtaining a sample.

4. The system of claim 2, wherein said warming table is configured to adjust one or more of temperature, height, orientation, distance from a subject, and presence or height of a rest.

5. The system of claim 3, wherein said calming feature is selected from the group of calming features consisting of flowing water, a flower, a plant, a calming scent, a calming sound, a calming image, and a calming color.

6. The system of claim 3, further comprising:
a bathroom adjacent said sample collection room; and
a pass-through connecting said sample collection room with said bathroom,
wherein said pass-through is configured to allow the transfer of a sample from said bathroom to said sample collection room.

7. (canceled)

8. The system of any of claim 3, further comprising one or more of the following:
a fingertoy;
Flowing water in said sample collection room;
Adjustable lighting in said sample collection room;
A calming image;
A wall or wall hanging having a calming color;
A sound system configured for providing calming sounds in the sample collection room; and
A scent source configured to provide a calming scent in the sample collection room, wherein said scent source is selected from a flower, a candle, an oil, a scented wick, and an aerosol.

9-11. (canceled)

12. A sample collection room in a retail store, said sample collection room being located adjacent one or more of a waiting room and a bathroom and comprising one or more of: a warming table, a warming plate, a chair or other furniture for seating comprising a warming plate, a finger warmer, a fingertip warmer, and an air-warmer.

13. The sample collection room in a retail store of claim 12, wherein said sample collection room is located adjacent to a sample analysis device or sample analysis system.

14. The sample collection room in a retail store of claim 12, further comprising a sample analysis device, a sample transport container configured to hold a plurality of sample containers, or both.

15. The sample collection room in a retail store of claim 14, wherein said sample analysis device is configured to analyze fluid samples having volumes of about 500 μL or less.

16. The sample collection room in a retail store of claim 14, wherein said sample containers are configured to hold fluid samples having volumes of about 500 μL or less.

17. (canceled)

18. The sample collection room in a retail store of claim 12, wherein said sample collection room is adjacent a bathroom, further comprising a pass-through connecting said bathroom with said sample collection room.

19. (canceled)

20. The sample collection room in a retail store of claim 12, wherein said warming device is configured to warm a body part of a subject effective to aid in the collection of a blood sample from said body part.

21. The sample collection room of claim 20, wherein said body part is selected from a fingertip, a finger, and a hand.

22-37. (canceled)

38. The system for obtaining a sample from a subject of claim 1, further comprising a fingertoy; a warming surface; a water feature; and a calming feature.

39. The system of claim 38, wherein said warming surface is configured to adjust one or more of temperature, height, orientation, distance from a subject, and presence or height of a rest configured to support a limb, hand, or foot of a subject.

40. The system of claim 38, further comprising:
a bathroom adjacent said sample collection room; and
a pass-through connecting said sample collection room with said bathroom,
wherein said pass-through is configured to allow the transfer of a sample from said bathroom to said sample collection room.

41. The system of claim 38, further comprising i) a sample analysis device, or ii) a sample transport container configured to hold a plurality of sample containers configured to hold fluid samples having volumes of about 500 μL or less, or both i) and ii).

42. The system of claim 38, wherein said sample is a blood or urine sample having a volume selected from the group of volumes of less than about 500 μL, or less than about 250 μL, or less than about 150 μL, or less than about 100 μL, or less than about 50 μL, or less than about 25 μL, or less than about 10 μL, or less than about 5 μL, or less than about 1 μL.

43-67. (canceled)
